(19) United States
(12) Patent Application Publication
(10) Pub. No.: US 2025/0134938 A1
(43) Pub. Date: May 1, 2025

(54) LACTOCOCCUS LACTIS SUBSP. LACTIS CAB701 HAVING ANTIOBESITY AND IMMUNE-PROMOTING ACTIVITIES AND USES THEREOF

(71) Applicant: BKBIO. CO., LTD., Jeju-do (KR)

(72) Inventors: Hyuk-Jun CHOI, Gyeonggi-do (KR); Un-Sik HWANG, Jeju-do (KR); Young-Seo PARK, Gyeonggi-do (KR); Hui-jin JEONG, Seoul (KR); Won-Kyu YOU, Chungcheongnam-do (KR)

(73) Assignee: BKBIO. CO., LTD., Jeju-do (KR)

(21) Appl. No.: 18/930,130

(22) Filed: Oct. 29, 2024

(30) Foreign Application Priority Data
Oct. 30, 2023 (KR) 102023-0146232
Oct. 28, 2024 (KR) 10-2024-0148704

(51) Int. Cl. A61K 35/744; A23C 11/10; A23L 33/135; A61P 3/04

(52) U.S. Cl. CPC A61K 35/744; A23C 11/10; A23L 33/135; A61P 3/04

(57) ABSTRACT

The present disclosure relates to a Lactococcus lactis subsp. lactis CAB701 strain having both anti-obesity and immune-promoting activities, an anti-obesity or immune-enhancing food composition containing the same, and an anti-obesity or immune-enhancing vegan fermented milk containing the same. The CAB701 strain of the present disclosure has pancreatic lipase inhibitory activity and can be widely utilized for preventing, ameliorating or treating obesity and for enhancing immunity. Furthermore, it can be used as a starter for fermentation. In addition, since a vegan fermented milk prepared with the CAB701 strain of the present disclosure exhibits a significantly slower decrease in the viable Lactobacillus cell count during the storage period as compared to vegan fermented milks prepared with conventional strains, it can be seen that the CAB701 strain of the present disclosure is useful in the preparation of a vegan fermented milk.

### BACKGROUND

### 1. Field

The present disclosure relates to a Lactococcus lactis subsp. lactis CAB701 strain having both anti-obesity and immune-promoting activities, an anti-obesity or immune-enhancing food composition containing the same, and an anti-obesity or immune-enhancing vegan fermented milk containing the same.

### 2. Description of the Related Art

As Korea's lifestyle has evolved into that of Western advanced countries along with economic development, changes in diet and decreased physical activity have led to an increase in the number of obese people due to increased body fat and metabolic imbalance, and the incidence of so-called metabolic syndrome, which is a complication of cardiovascular diseases such as diabetes, hyperlipidemia, thrombotic disorder, etc., is increasing continuously (Lee S J, Park J Y, Nam C M, Jee S H. 2008. The prevalence estimation of metabolic syndrome and its related factors based on data from general health medical examination: a multi-center study. J Korean Soc Health Information Health Statistics 33:119-133; Frohlich J, Lear S A. 2002. Old and new risk factors for atherosclerosis and development of treatment recommendations. Clin Exp Pharmacol Physiol 29:838-842).

Obesity is not a disease caused by a single cause, but a syndrome that is caused by a combination of genetic, environmental and social factors, and is caused by excess energy intake that is not consumed in the body's metabolism and accumulated as triglycerides in adipose tissue (Chua, S. C. Jr. Monogenic models of obesity. Behav Genet. 27:277-284, 1997). In addition, it is reported that the increased blood fatty acid concentration and abdominal fat seen in obesity are common features of hyperlipidemia and type 2 diabetes, and the increase in free fatty acids leads to ventricular hypertrophy (DeFronzo, R. A, Ferrannini, E. Insulin resistance. A multifaceted syndrome responsible for NIDDM, obesity, hypertension, dyslipidemia, and atherosclerotic cardiovascular disease. Diabetes Care. 14 (3): 173-194, 1991), and that abdominal fat accumulation is closely associated with the development of insulin resistance (Tamori, Y., Kasuga, M. Obesity and insulin resistance. Nippon Rinsho. 67 (2): 236-244, 2009), which causes or exacerbates lifestyle diseases such as hypertension, atherosclerosis, diabetes, hyperlipidemia, etc.

While it is important to prevent health problems caused by obesity, long-term, effective obesity management and treatment are need for patients. Currently available methods for treating obesity include lifestyle modifications such as diet, exercise and behavioral therapies, as well as medication and surgical treatment (Mason E E. 1992. Methods for voluntary weight loss and control. Obes Surg 2:275-276). In general, lifestyle modification alone is not enough to treat obesity, so in many cases, medication is needed in addition to lifestyle modification. The use of medications to treat obesity has been tried from long ago, but due to a number of serious side effects, the two drugs currently approved for long-term use by the U.S. Food and Drug Administration (FDA) are sibutramine, which works by inhibiting the reuptake of norepinephrine and serotonin, and orlistat, which works by inhibiting lipases secreted by the pancreas and digestive system (King D J, Devaney N. 1988. Clinical pharmacology of sibutramine hydrochloride (BTS 54524), a new antidepressant, in healthy volunteers. Br J Pharmacol 26:607-611; Yanovski S Z, Yanovski J A: Obesity. N Engl J Med 346:591-602, 2002). However, both of these drugs are not widely used due to their side effects (Padwal R, Li S K, Lau D C: Long-term pharmacotherapy for overweight and obesity: a systematic review and meta-analysis of randomized controlled trials. Int J Obes Relat Metab Disord 27:1437-1446, 2003; Thearle M, Aronne L J: Obesity and pharmacologic therapy: Endocrin Metab Clin North Am 32:1005-24, 2003).

Triglycerides in food are hydrolyzed into fatty acids and monoglycerides by the action of pancreatic lipase, and the resulting fatty acids form micelles with cholesterol. As a result, the fatty acids are absorbed by the body in the form of micelles. Lipase is a type of esterase that breaks down ingested lipids into fatty acids and glycerol. There are gastric lipases, pancreatic lipases, etc. in the human body. The digestion of fats is rarely accomplished by the gastric lipases but is mostly accomplished in the intestinal tract by the pancreatic lipases (Goldstein J. L., Schrott H., Hazzard E., Bierman E., Motuski A., J. Clin. Invest., 52, 1544-1568 (1973); Rodwell V. W., McNamara D. J., Shapiro D. J., Adv. Enzymol., 38, 373-412 (1973); Carey M. C., Small D. M., Bliss C. M., Annu. Rev. Physiol., 45, 651-677 (1983)).

Lipase inhibitors and lipase-inhibiting compositions are enteric lipase inhibitors that inhibit lipases in the intestinal tract. When the lipases are inactivated, they are unable to hydrolyze dietary fats into absorbable monoglycerides and free fatty acids. The triglycerides that are not hydrolyzed are not absorbed by the body, resulting in calorie reduction. Therefore, they can be used in the treatment of obesity and can be used in conjunction with a low-calorie diet to reduce and maintain body weight. In particular, the inhibition of lipase is important for the absorption of fatty substances such as triglycerides and cholesterol in the blood. In particular, reducing fat in the blood is important for the prevention and treatment of cardiovascular diseases, since increased fat in the blood is known to contribute to cardiovascular diseases. Hyperlipidemia, an increased level of lipids in the blood, is caused by excessive intake of cholesterol, triglycerides, etc. and abnormalities in lipoprotein metabolism, such as increased biosynthesis and decreased degradation of lipoproteins, or delayed clearance of lipoproteins from the plasma. In particular, hypercholesterolemia contributes to ischemic heart disease and arteriosclerosis (Kwang Woo Lee, Diabetes and Obesity. Diabetes, 14 (1) 5-11 (1990); Hong Kyu Lee, Obesity and Related Diseases. Korean Journal of Obesity, 1 (1), 34-39 (1992); Min-Young Jung, Comorbidities of Obesity. Korean Journal of Obesity, 1 (1), 5-10 (1992); Clinical Obesity, Korean Society for the Study of Obesity. Koryo Medicine. 281-283 (1995)).

Therefore, research on the development of lipase inhibitors is of medical and nutritional importance, and many researchers are focusing on the development of lipase inhibitors derived from natural products (N. B. Cater and S. M. Grundy, International Journal of Obesity., 3 (35), 11 (1987)). Satouchi et al. isolated a protein with lipase inhibitory activity from soybean (K. Satouchi, T. Mori and S. Matsushita: Characterization of Inhibitor Protein for lipase in Soybean Seeds. Agr. Biol. Chem., 38 (1), 97-101 (1974)). In Korea, Kim et al. searched for lipase inhibitory activity from pimento (Kim. B. M.: Lipase Inhibitors from green pepper Capsocum annuun Lin. Korean J. Food Sci. Technol., 9 (3), 234-240 (1997)), and Shimura et al. reported that a tannin isolated from fleabane (senna) inhibited lipase activity (S. Shimura, W. Tsuzuki, S. Kobayashi and T. Suzuki: Nippon Shokuhin Kogyo Gakkaishi., 41 (8), 561-564 (1994)).

The inventors of the present disclosure have made research efforts to develop a pancreatic lipase inhibitor. As a result, they have found that a Lactococcus lactis subsp. lactis CAB701 strain isolated from cabbage has excellent pancreatic lipase inhibitory activity, and furthermore has both anti-obesity and immune-enhancing activities, and have completed the present disclosure.

### SUMMARY

The present disclosure is directed to providing a Lactococcus lactis subsp. lactis CAB701 strain having both anti-obesity and immune-enhancing activities.

The present disclosure is also directed to providing an anti-obesity or immune-enhancing food composition, which contains a Lactococcus lactis subsp. lactis CAB701 strain as an active ingredient.

The present disclosure is also directed to providing an anti-obesity or immune-enhancing vegan fermented milk, which contains a Lactococcus lactis subsp. lactis CAB701 strain.

The present disclosure is also directed to providing a method for preparing an anti-obesity or immune-enhancing vegan fermented milk, which includes a step of fermenting a plant-based milk substitute with a Lactococcus lactis subsp. lactis CAB701 strain.

The present disclosure provides a Lactococcus lactis subsp. lactis CAB701 strain with an accession number KCCM 13360P, which has both anti-obesity and immune-enhancing activities.

According to an exemplary embodiment of the present disclosure, the strain may be acid-resistant to pH 2-3, bile-resistant to 0.1-1% bile acid and pancreatic juice-resistant to 0.1-1% pancreatic juice, and may have the ability of intestinal adherence.

According to an exemplary embodiment of the present disclosure, the strain may have lipase inhibitory activity.

According to an exemplary embodiment of the present disclosure, the strain may be one that promotes the production of nitric oxide (NO), which is an immune marker.

According to an exemplary embodiment of the present disclosure, the strain may contain a 16S rRNA base sequence represented by SEQ ID NO 1.

The present disclosure also provides an anti-obesity or immune-enhancing food composition, which contains a Lactococcus lactis subsp. lactis CAB701 strain (accession no: KCCM 13360P), a culture of the strain, a supernatant of the culture, a concentrate of the culture, a dried product of the culture, a lysate of the strain, or a fermentation product of the strain as an active ingredient.

According to an exemplary embodiment of the present disclosure, the composition may promote the production of nitric oxide (NO), which is an immune marker.

According to an exemplary embodiment of the present disclosure, the composition may increase the expression of the TNF-α, IL-6, IL-1β, COX-2 or iNOS gene, which are immune markers.

According to an exemplary embodiment of the present disclosure, the composition may inhibit lipase activity.

According to an exemplary embodiment of the present disclosure, the composition may be for preventing or ameliorating a disease caused by decreased immune function.

According to an exemplary embodiment of the present disclosure, the disease caused by decreased immune function may be one or more selected from an infectious disease, an allergic disease and chronic fatigue.

The present disclosure also provides an anti-obesity or immune-enhancing vegan fermented milk, which contains a Lactococcus lactis subsp. lactis CAB701 strain (accession no: KCCM 13360P), a culture of the strain, a supernatant of the culture, a concentrate of the culture, a dried product of the culture, a lysate of the strain, or a fermented product of the strain.

The present disclosure also provides a method for preparing an anti-obesity or immune-enhancing vegan fermented milk, which includes: (1) a step of inoculating a plant-based milk substitute with a Lactococcus lactis subsp. lactis CAB701 strain (accession no: KCCM 13360P) at 0.2 to 3% (v/v); and (2) a step of fermenting the plant-based milk substitute inoculated with the strain at 35 to 39° C. for 12 to 24 hours to pH 3.5 to 4.5.

The CAB701 strain of the present disclosure has pancreatic lipase inhibitory activity and can be widely utilized for preventing, ameliorating or treating obesity and for enhancing immunity. Furthermore, it can be used as a starter for fermentation.

In addition, since a vegan fermented milk prepared with the CAB701 strain of the present disclosure exhibits a significantly slower decrease in the viable Lactobacillus cell count during the storage period as compared to vegan fermented milks prepared with conventional strains, it can be seen that the CAB701 strain of the present disclosure is useful in the preparation of a vegan fermented milk.

### BRIEF DESCRIPTION OF THE DRAWINGS

FIG. 1 is a phylogenetic tree of a CAB701 strain isolated in an example of the present disclosure.

FIGS. 2A and 2B are genome maps of a L. lactis subsp. lactis strain according to an exemplary embodiment of the present disclosure. FIG. 2A shows the circular chromosome and plasmid of the CAB701 strain, and FIG. 2B shows the chromosome of the WiKim0124 strain. In FIGS. 2A and 2B, the outermost band represents contigs, the second and third bands represent COG functions, the fourth band represents rRNAs and tRNAs in the genome, and the fifth band represents the GC skew metric as an indicator for identifying replication loci and leading or lagging strands (green: higher-than-average values; red: lower-than-average values). The innermost band represents the GC ratio (blue: higher-than-average values; yellow: lower-than-average values).

FIG. 3 illustrates the CAZy classification (prediction of genes involved in carbohydrate metabolism) of L. lactis strains through predictive analysis of gene function annotation according to an exemplary embodiment of the present disclosure.

FIG. 4 is a heat map depicting the phylogenetic relationship of L. lactis strains according to an exemplary embodiment of the present disclosure.

FIG. 5 is a graph depicting the genome alignment of the CAB701 strain and the WiKim0124 strain using NUCMER according to an exemplary embodiment of the present disclosure.

FIG. 6 is a graph depicting the genome alignment of the CAB701 strain and the WiKim0124 strain using Mauve according to an exemplary embodiment of the present disclosure.

FIG. 7 is a Venn diagram showing the result of pan-genome analysis of L. lactis strains according to an exemplary embodiment of the present disclosure.

FIG. 8 shows Pareto charts depicting the standardized effect of various factors (sodium acetate, citric acid, manganese sulfate, yeast extract, magnesium sulfate, potassium phosphate, sucrose and polysorbate 80) affecting the growth of the CAB701 strain according to an exemplary embodiment of the present disclosure. In FIG. 8, the response is expressed as the number of living cells (log CFU/mL).

FIGS. 9A and 9B show three-dimensional (FIG. 9A) and contour (FIG. 9B) plots of RSM showing the combined effect of four variables (yeast extract, citric acid, sodium acetate and manganese sulfate) on the number of viable cells of the CAB701 strain according to an exemplary embodiment of the present disclosure.

FIG. 10 is a growth curve of the CAB701 strain according to an exemplary embodiment of the present disclosure depending on sucrose concentration.

FIGS. 11A and 11B show images confirming the differentiation of 3T3-L1 cells treated with a CAB701 strain isolated in an example of the present disclosure to adipocytes (FIG. 11A), and graphs showing the rate of adipocyte differentiation (FIG. 11B). In FIGS. 11A and 11B, “MRS” refers to 3T3-L1 preadipocytes exposed to the CAB701 strain cultured in a non-optimized MRS medium, and “YSC” refers to 3T3-L1 preadipocytes exposed to the CAB701 strain cultured in an optimized medium of the present disclosure (YSC medium).

FIGS. 12A to 12C show the change in the number of living cells and pH over time for the CAB701 strain according to an exemplary embodiment of the present disclosure (FIG. 12A), and three-dimensional (FIG. 12C) and contour (FIG. 12B) plots of RSM showing the combined effect of three variables (initial pH, temperature and time) on the number of living cells.

FIG. 13 is a graph showing the cytotoxicity of the CAB701 strain isolated in an example of the present disclosure against RAW 264.7 cells.

FIG. 14 shows graphs depicting the expression level of cytokine-related genes (INOS, COX-2 and TNF-α) in RAW 264.7 cells treated with the CAB701 strain isolated in an example of the present disclosure.

FIG. 15 shows graphs depicting the expression level of MAPKs signaling pathway-related proteins in RAW 264.7 cells treated with the CAB701 strain isolated in an example of the present disclosure.

FIG. 16 is a graph depicting the cytotoxicity of the CAB701 strain isolated in an example of the present disclosure against 3T3-L1 preadipocytes.

FIG. 17A shows images confirming the differentiation of 3T3-L1 cells treated with the CAB701 strain isolated in an example of the present disclosure to adipocytes, and FIG. 17B is a graph showing the rate of adipocyte differentiation.

FIG. 18 shows graphs depicting the expression level of adipogenesis-related genes or fat metabolism-related genes in 3T3-L1 cells treated with the CAB701 strain isolated in an example of the present disclosure.

FIG. 19 shows graphs depicting the expression level of fat metabolism-related proteins in 3T3-L1 cells treated with the CAB701 strain isolated in an example of the present disclosure.

FIG. 20 shows E-test images showing the antibiotic resistance of the CAB701 strain isolated in an example of the present disclosure.

FIG. 21 shows a result of analyzing the enzymatic activity of the CAB701 strain isolated in an example of the present disclosure using an API ZYM kit.

FIG. 22 shows graphs depicting the change in body weight, weight gain and periepididymal adipose tissue weight of mice administered with the CAB701 strain isolated in an example of the present disclosure for 6 weeks together with a high-fat diet.

FIG. 23 shows graphs depicting serum biochemical markers (triglycerides, total cholesterol, HDL-cholesterol and LDL-cholesterol) of mice administered with the CAB701 strain isolated in an example of the present disclosure for 6 weeks together with a high-fat diet.

FIG. 24 shows graphs depicting the expression level of lipogenic factor proteins (FAS and PPAR-y) in the periepididymal adipose tissue of mice administered with the CAB701 strain isolated in an example of the present disclosure for 6 weeks together with a high-fat diet.

FIG. 25 shows images of depicting the result of staining periepididymal adipose tissues of mice administered with the CAB701 strain isolated in an example of the present disclosure for 6 weeks together with a high-fat diet with H&E and observing adipocyte size under an optical microscope.

FIG. 26 is a graph depicting the result of staining periepididymal adipose tissues of mice administered with the CAB701 strain isolated in an example of the present disclosure for 6 weeks together with a high-fat diet with H&E and observing adipocyte size under an optical microscope.

### DETAILED DESCRIPTION

The present disclosure will now be described in detail.

In the present disclosure, “obesity” refers to a condition in which excess body fat has accumulated, rather than a state of simply being overweight. It means that one with a high body fat percentage can be obese, even if he/she appears to be of normal body weight. Usually, body mass index (BMI) is used to evaluate obesity: 23 to 24.9 is considered overweight, 25 to 29.9 as mild obesity, 30 to 34.9 as moderate obesity, and 35 or higher as severe obesity. Obesity is caused by a combination of factors rather than a single cause, including poor dietary habits such as westernized eating habits and decreased physical activities, emotional factors, genetics factors, etc. The obesity increases the risk of the development of chronic diseases such as hyperlipidemia, diabetes, hypertension, etc.

As used herein, “immune enhancement” means a function that promotes an immune response to an antigen non-specifically during the initial activation of immune cells, or a function that enhances immunity by increasing the activity of cells of the immune system.

The present disclosure relates to a Lactococcus lactis subsp. lactis CAB701 strain with an accession number KCCM 13360P, which has both anti-obesity and immune-enhancing activities.

The Lactococcus lactis subsp. lactis CAB701 strain of the present disclosure is a novel Lactococcus lactis subsp. lactis strain derived from cabbage. Although the Lactococcus lactis subsp. lactis CAB701 strain of the present disclosure is isolated from cabbage, there is no limitation in the source.

It was identified in an example that the isolated Lactobacillus strain has a base sequence of SEQ ID NO 1 as determined by 16S rRNA sequencing for identification and classification of microorganisms.

Accordingly, the microorganism of the present disclosure having the 16S rRNA base sequence of SEQ ID NO 1 was named a Lactococcus lactis subsp. lactis CAB701 strain and deposited in the Korean Culture Center of Microorganisms on Jun. 16, 2023 (accession number KCCM 13360P).

The Lactococcus lactis subsp. lactis CAB701 strain of the present disclosure is a gram-positive facultative anaerobe capable of growing under both aerobic and anaerobic conditions. It does not form spores and is nonmotile, and its cells are cocci.

According to experimental results of the present disclosure, short-term or long-term administration of a high-fat diet significantly increased body weight and body fat. In addition, it increased blood triglycerides, blood total cholesterol, and blood LDL cholesterol concentrations, leading to obesity.

In an example, high immune-enhancing activity was confirmed since treatment of RAW 264.7 cells with the Lactococcus lactis subsp. lactis CAB701 strain of the present disclosure resulted in increased NO production to 86.32% of an LPS-treated positive control group.

In another example, excellent immune-enhancing activity was confirmed since treatment of RAW 264.7 cells with the CAB701 strain of the present disclosure resulted in significant increase in the expression of cytokine-related genes (iNOS, COX-2, TNF-α, IL-1β and IL-6) and the expression of proteins related to the MAPKs signaling pathway.

In another example, as a result of investigating the pancreatic lipase inhibition rate of the CAB701 strain of the present disclosure, superior pancreatic lipase inhibition was confirmed since the pancreatic lipase inhibition rate (28.87%) was the same as that of orlistat (1000 μg/mL), which is a lipase inhibitor.

In another example, as a result of culturing mouse preadipocytes (3T3-L1) in an adipogenic differentiation medium to induce differentiation into adipocytes, treating them with the CAB701 strain and investigating the extent of adipocyte differentiation using the Oil Red O staining method to determine the anti-obesity effect of the CAB701 strain, a group in which the differentiation into adipocytes was not induced (CON) showed no staining, and all the test groups treated with the CAB701 strain showed decreased staining in red color in a concentration-dependent manner, indicating that the differentiation into adipocytes was inhibited by 61.47%.

In another example, as a result of investigating the inhibitory effect of the CAB701 strain on the expression of genes involved in lipogenesis using preadipocytes, it was found that the expression of the LPL and adiponectin genes, which are important transcription factors for differentiation into adipocytes, was significantly reduced in a CAB701 strain-treated group as compared to a positive control group treated only with a differentiation medium without treating with the CAB701 strain.

In another example, a normal diet was given to mice of a normal group (NOR), only a high-fat diet (HFD) was given to an obesity-induced group, and the CAB701 strain was administered orally while giving a high-fat diet to a CAB701 administration group. The mice were then examined for body weight and adipose tissue weight. As a result, it was found that the CAB701 administration group showed significantly reduced body weight and adipose tissue weight. In addition, it was confirmed that blood triglycerides, total cholesterol and LDL-cholesterol concentrations were decreased by 56.76%, 81.94% and 72.94%, respectively, in the CAB701 administration group as compared to an obesity control group. In addition, it was confirmed specifically that the adipocyte size in periepididymal adipose tissue was decreased to 70.19% in a CAB701 administration group as compared to an obesity control group.

Thus, it can be seen that the CAB701 strain of the present disclosure possesses pancreatic lipase activity and simultaneously has anti-obesity and immune-enhancing activities.

Furthermore, the present disclosure relates to an anti-obesity or immune-enhancing food composition, which contains a Lactococcus lactis subsp. lactis CAB701 strain (accession no: KCCM 13360P), a culture of the strain, a supernatant of the culture, a concentrate of the culture, a dried product of the culture, a lysate of the strain or a fermentation product of the strain as an active ingredient.

Since the “Lactococcus lactis subsp. lactis CAB701 strain”, “anti-obesity” and “immune enhancement” of the present disclosure have already been described above, the description thereof will be omitted to avoid excessive redundancy.

As used herein, “as an active ingredient” or “containing as an active ingredient” means containing an amount sufficient to achieve the efficacy or activity of the CAB701 strain of the present disclosure and, more specifically, comprising an amount sufficient to exhibit anti-obesity or immune-enhancing activity. In an exemplary embodiment, the CAB701 strain may be used at a concentration of 10 to 15,000 mg/kg, specifically 100 to 10,000 mg/kg. In another exemplary embodiment, the CAB701 strain may be used at a concentration of 1×10⁶ CFU/mL to 1×10¹⁰ CFU/mL, specifically 1×10⁸ CFU/mL to 1×10¹⁰ CFU/mL. Since the CAB701 strain is a natural product and has no adverse effects on the human body even when administered in excess, the quantitative upper and lower limits of the CAB701 strain contained in the composition of the present disclosure can be selected by those skilled in the art within an appropriate range.

The food composition of the present disclosure can be formulated and utilized as a functional food or may be added to various food products. The food products to which the composition of the present disclosure can be added include, for example, beverages, alcoholic beverages, confectionery, diet bars, dairy products, meat, chocolate, pizza, ramen, other noodles, chewing gum, ice cream, vitamin complexes, dietary supplements, etc.

The food composition of the present disclosure may contain, not only the CAB701 strain as an active ingredient, but also ingredients customarily added in food preparation, including, for example, proteins, carbohydrates, fats, nutrients, seasonings, and flavorings. Examples of the carbohydrate include: common sugars such as monosaccharides, e.g., glucose, fructose, etc.; disaccharides, e.g., maltose, sucrose, etc.; oligosaccharides; polysaccharides, e.g., dextrin, cyclodextrin, etc., and sugar alcohols such as xylitol, sorbitol, erythritol, etc. As the flavoring, natural flavorings [thaumatin or stevia extract (e.g., rebaudioside A, glycyrrhizin, etc.)] and synthetic flavorings (saccharin, aspartame, etc.) can be used. For example, when the food composition of the present disclosure is formulated as a drink or a beverage, it may further contain, in addition to the CAB701 strain of the present disclosure, citric acid, high-fructose corn syrup, sugar, glucose, acetic acid, malic acid, fruit juice, various plant extracts, etc.

The present disclosure provides a health functional food, which contains the anti-obesity and immune-enhancing food composition containing the CAB701 strain as an active ingredient. The health functional food means a food product prepared by adding the CAB701 strain to food materials such as beverages, teas, spices, chewing gum, confectionery, etc. or preparing it into a capsule, a powder, a suspension, etc., which brings specific health effects when consumed, but unlike ordinary drugs, is advantageous in that it has no side effect that may occur when taking drugs for a long time. The health functional food of the present disclosure obtained in this way is very useful because it can be consumed on a daily basis. The amount of the CAB701 strain added to the health functional food cannot be uniformly prescribed as it depends on the type of the health functional food. But, it can be added in a range that does not impair the original flavor of the food, typically in a range of 0.01 to 50 wt %, specifically 0.1 to 20 wt %, of the food. Furthermore, when the health functional food is in the form of a powder, a granule, a tablet or a capsule, the addition amount is typically in a range of 0.1 to 100 wt %, specifically 0.5 to 80 wt %. In a specific exemplary embodiment, the health functional food of the present disclosure may be in the form of a pill, a tablet, a capsule or a beverage.

Furthermore, the present disclosure relates to an anti-obesity or immune-enhancing vegan fermented milk, which contains a Lactococcus lactis subsp. lactis CAB701 strain (accession no: KCCM 13360P), a culture of the strain, a supernatant of the culture, a concentrate of the culture, a dried product of the culture, a lysate of the strain, or a fermentation product of the strain.

Since the “Lactococcus lactis subsp. lactis CAB701 strain”, “anti-obesity” and “immune enhancement” of the present disclosure have already been described above, the description thereof will be omitted to avoid excessive redundancy.

The vegan fermented milk of the present disclosure not only possesses anti-obesity and immune-enhancing activities due to fermentation using the CAB701 strain, but also has excellent storage properties since the decrease in the viable Lactobacillus cell count during the storage period is significantly slower as compared to vegan fermented milks prepared using conventional vegan fermented milk strains.

The present disclosure also provides a method for preparing an anti-obesity or immune-enhancing vegan fermented milk, which includes: (1) a step of inoculating a plant-based milk substitute with a Lactococcus lactis subsp. lactis CAB701 strain (accession no: KCCM 13360P) at 0.2 to 3% (v/v); and (2) a step of fermenting the plant-based milk substitute inoculated with the strain to pH 3.5 to 4.5 at 35 to 39° C. for 12 to 24 hours.

Since the “Lactococcus lactis subsp. lactis CAB701 strain”, “anti-obesity” and “immune enhancement” of the present disclosure have already been described above, the description thereof will be omitted to avoid excessive redundancy.

The plant-based milk substitute may be a milk substitute prepared from one or more plant-based raw material selected from coconut, soybean, almond, oats and rice.

The plant-based milk substitute may have a solid content of 4 to 15 wt %, specifically 5 to 10 wt %, more specifically 7 to 9 wt %.

The vegan fermented milk may be stored at 1 to 8° C., specifically at 2 to 6° C., after preparation.

The vegan fermented milk may have a live bacterial count of 130×10⁶ CFU/mL or larger, specifically 130×10⁶ CFU/mL to 150×10⁶ CFU/mL, immediately after preparation, and may maintain a live bacterial count of 70×10⁶ CFU/mL or larger, specifically 70×10⁶ CFU/mL to 120×10⁶ CFU/mL, more specifically 90×10⁶ CFU/mL to 120×10⁶ CFU/mL, until 14 days after the preparation, or the expiration date, when stored at 1 to 8° C., specifically 2 to 6° C.

### Example 1: Isolation and Identification of Strains

1-1: Screening of Lactobacilli from Korean Plant Sources

For isolation of Lactobacilli derived from Korean plant resources, 8 types of Jeju raw materials provided by BK Bio were used. The Jeju raw materials used for the isolation of Lactobacilli are as follows.
Jeju raw materials (8 types): carrot, cactus, broccoli, beet, cabbage, hallabong, kale and kohlrabi

Specifically, after adding appropriately cut Jeju raw materials and 0.85% (w/v) NaCl (sterile physiological saline) in a Stomacher bag, the samples were homogenized. The homogenized samples were diluted decimally with sterile physiological saline, and then the 100-, 10-1- and 10-2-fold diluted samples were smeared on bromocresol purple (BCP)-MRS agar medium and incubated anaerobically at 37° C. for 20 hours. For each sample, 10 colonies showing yellow color were subcultured for 2 passages in BCP-MRS.

### 1-2: Isolation and Identification of Strains

### 1-1-1: Isolation of Strains

Through morphological and physiological examination of the putative lactic acid bacteria in the BCP-MRS medium, a cabbage-derived strain that was gram-positive and catalase-negative, and possessed both anti-obesity and immune-enhancing activities was isolated finally. The isolated strain was subcultured in MRS medium for at least 2 passages before being used in experiments.

### 1-1-2: Identification of Strains

(1) The finally selected strain was identified by 16S rRNA gene sequencing. For the isolation of the genomic DNA of each strain, colonies formed on the MRS agar plate were inoculated onto MRS broth medium and cultured at 37° C. for 16 hours. Then, 3 mL of the culture was centrifuged at 13,000 rpm and 4° C. for 1 minute to harvest the strain. DNA was extracted from the gram-positive bacteria with a DNA extraction kit (AccuPrep® genomic DNA extraction kit, Bioneer, Daejeon, Korea) using a lysis buffer (20 mM Tris-HCl (pH 8.0), 2 mM EDTA, 1.2% Triton® X-100) and lysozyme (60 mg/mL) according to the manufacturer's protocol. PCR amplification for identification was performed using a PCR premix (AccuPower@ PCR PreMix, Bioneer) according to the manufacturer's protocol. The PCR reaction was performed using 20 ng of template DNA, a 27F primer (5′-AGA GTT TGA TCA TGG CTC AG-3′), a 1492R primer (5′-TAC GGY TAC CTT GTT ACG AC-3′), and a thermocycler (TurboCycler, Blue-Ray Biotech, Taipei, Taiwan) under the conditions described in Table 1 below. The PCR-amplified DNA was purified using a QIAquick® PCR purification kit (Qiagen, Hilden, Germany) and sequenced by the Sanger sequencing method at Bionics, Inc. The 16S rRNA gene sequences were compared using BLAST (basic local alignment search tool) available at the National Institutes of Health (NCBI, Bethesda, MD, USA).

TABLE 1

| Step | Temperature (° C.) | Time (sec) | Number of cycles |
|---|---|---|---|
| Pre-denaturation | 94 | 120 | 1 |
| Denaturation | 94 | 30 | 35 |
| Annealing | 50 | 60 | 35 |
| Extension | 72 | 60 | 35 |
| Post-extension | 72 | 490 | 1 |

(2) As a result of the 16S rRNA gene sequencing, the strain isolated from cabbage among the Jeju raw materials, which possessed both anti-obesity and immune-enhancing activities, was identified as a Lactococcus lactis subsp. lactis CAB701 strain.

### (3) Phylogenetic Analysis

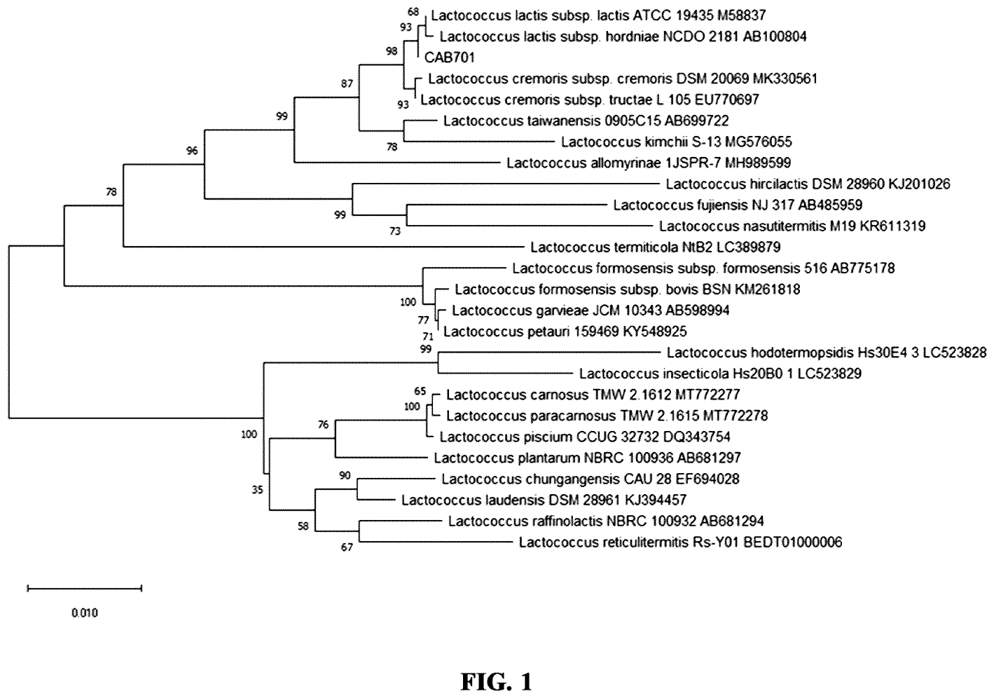

Phylogenetic analysis was performed on the Lactococcus lactis subsp. lactis CAB701 strain. Lactococcus includes 22 genera and 6 subspecies. The 16S rRNA gene sequences of the type strains were obtained from the LPSN (list of prokaryotic names with standing in nomenclature genus, http://www.bacterio.net). The sequences were aligned and edited using BioEdit (Ibis Biosciences, http://www.mbio.ncsu.edu/bioedit/bioedit.html) and ClustalW (http://clustal.org). A phylogenetic tree for the CAB701 strain was generated using MEGA11 (https://www.megasoftware.net) by the neighbor-joining method (FIG. 1).

### (4) Analysis of Sugar Availability

Sugar availability was analyzed for the Lactococcus lactis subsp. lactis CAB701 strain using an API20 STREP kit.

TABLE 2

| Sugar | CAB701 |
|---|---|
| Glycerol | − |
| Erythritol | − |
| D-Arabinose | − |
| L-Arabinose | − |
| D-Ribose | + |
| D-Xylose | + |
| L-Xlose | + |
| D-Adonitol | − |
| Methyl-β-D-xylopyranoside | − |
| D-Galactose | − |
| D-Glucose | + |
| D-Fructose | + |
| D-Mannose | + |
| L-Sorbose | + |
| L-Rhamnose | − |
| Dulcitol | − |
| Inositol | − |
| D-Mannitol | − |
| D-Sorbitol | + |
| Methyl-α-D-mannopyranoside | − |
| Methyl-α-D-glucopyranoside | − |
| N-Acetylglucosamine | − |
| Amygdalin | + |
| Arbutin | + |
| Esculin ferric citrate | + |
| Salicin | + |
| D-Cellobiose | + |
| D-Maltose | + |
| D-Lactose (bovine origin) | + |
| D-Melibiose | − |
| D-Saccharose (sucrose) | + |
| D-Trehalose | + |
| Inulin | + |
| D-Melezitose | − |
| D-Raffinose | − |
| Amidon (starch) | + |
| Glycogen | − |
| Xylitol | − |
| Gentiobiose | + |
| D-Turanose | − |
| D-Lyxose | − |
| D-Tagatose | − |
| D-Fucose | − |
| L-Fucose | − |
| D-Arabitol | − |
| L-Arabitol | − |
| Potassium gluconate | + |
| Potassium 2-ketogluconate | − |
| Potassium 5-ketogluconate | − |

### Example 2: Genomic Analysis

### Experimental Methods

### (1) Genomic DNA Sequencing, Assembly and Annotation Methods

### 5 DNA Extraction and Quality Assessment

Genomic DNA integrity was verified by agarose gel electrophoresis. The concentration of the extracted genomic DNA was quantified using a Qubit 2.0 fluorometer (Invitrogen, Carlsbad, USA). To ensure the purity and integrity of the genomic DNA, the 16S rRNA gene was sequenced using an ABI 3730 DNA sequencing system (Applied Biosystems, Foster City, CA, USA). The specifications for the DNA sample quantity and quality include concentration ≥10 ng/μL, total amount 400 ng, and A260/A280 ratio ≥1.8.

### Construction of MiSeq Library

For library construction, the genomic DNA was fragmented to approximately 550 bp using an M220 Focused-ultrasonicator™ (Covaris Ltd, Brighton, UK). The fragmented DNA was quantified using Bioanalyzer 2100 (Agilent, Palo Alto, USA) and a DNA 7500 kit. Then, a library was constructed using a TruSeq DNA library LT Kit (Illumina, San Diego, USA) according to the manufacturer's protocol. Whole genome sequencing was performed on an Illumina MiSeq system using 2×300 bp paired-end reads and a 600-cycle sequencing kit (MiSeq Reagent Kit v3).

### Construction of PacBio Library

For library construction, 5 μg of the genomic DNA was sheared using Megaruptor3 according to the manufacturer's recommended protocol and purified with AMpureXP beads to remove small fragments. Then, an SMRTbell library was constructed using an SMRTbell® Express template preparation kit (100-938-900). Genome size, library size and Qubit concentration were calculated using a PacBio calculator, and the library was pooled accordingly. DNA fragments smaller than 3 kb were removed using AMpure XP beads. For sequencing, an SMRTbell template was prepared by annealing a sequencing primer v4 and ligating with a DNA polymerase using a sequel binding kit 3.0. Excess primers and polymerase were removed by AMPure purification prior to the sequencing. The SMRTbell library was sequenced on the sequel system using a sequencing kit v3.0 and SMRT Cell 1M v2, and data were collected from 10 hours of video per the SMRT Cell 1M v2.

### Whole Genome Sequencing and Assembly

The genome of the target strain was constructed de novo using both the PacBio and MiSeq sequencing data. Sequencing analysis was performed by CJ Bioscience Inc. (Seoul, Korea). The MiSeq sequencing data was quality-controlled with Trimmomatic-0.36, and PhiX sequences were removed with BBMap 38.32. Hybrid assembly was achieved using Unicycler v 0.4.9, which incorporated the quality-controlled MiSeq data and PacBio long reads. If the resulting contigs from the hybrid assembly were circularized, they were rearranged to start at the dnaA/repA gene or the origin of replication using Circlator 1.4.0.

### Gene Prediction and Annotation

The gene-finding and functional annotation pipeline for whole-genome assembly was used from the EzBioCloud genome database. Protein-coding sequences (CDS) were predicted with Prodigal 2.6.2. tRNA genes were identified using tRNAscan-SE 1.3.1, and rRNAs and other non-coding RNAs were identified using covariance model search in the Rfam 12.0 database. CRISPR was detected using PilerCR 1.06 and CRT 1.2. The CDS were classified into groups based on their roles, with reference to orthologous groups in EggNOG 4.5. For further functional annotation, the genome sequence was analyzed using the UBLAST program to compare the predicted CDS with databases such as Swissprot (UniProt 2015), KEGG and SEED. To extend the functional classification, each CDS was aligned to the COG database using BLASTp with an e-value cutoff of 1×10⁻³. The EggNOG mapper tool was used for comprehensive functional annotation, identification of orthologous groups, and prediction of functional annotations based on conserved protein domains. The dbCAN3 meta-server was utilized for automated CAZy family annotation for prediction of carbohydrate-active enzymes.

### (2) Comparative Genomics

### Data Selection

For comparative genomics, the complete genome sequences of strains closely related to L. lactis subsp. lactis CAB701 were retrieved from the NCBI GenBank database. The strains selected by searching the database are L. lactis subsp. lactis ATCC 19435, L. lactis subsp. hordniae NBRC 100931, L. cremoris (formerly lactis subsp. cremoris) LMG 6897, and L. cremoris subsp. tructae (formerly lactis subsp. tructae) DSM 21502. In addition, the genome of L. lactis subsp. lactis Wikim0124 was selected based on phylogenetic proximity and functional relevance. The genome sequences of the selected strains were obtained using the EzBioCloud database (Yoon et al., 2017). Comprehensive comparative genomic analysis was performed using a comparative genomic analysis tool provided by CJ Bioscience Inc. (https://www.ezbiocloud.net/contents/cg).

### Phylogenetic Analysis

OrthoANI values were calculated to reveal the phylogenetic relationship between the analyzed strains. The orthologous ANI tool (OAT) (I. Lee, Kim, Park & Chun, 2016) provided by CJ Bioscience was used to generate an UPGMA (unweighted pair group method with arithmetic mean) dendrogram with arithmetic mean, which clearly visualized the evolutionary distance between strains.

### Genome Alignment and Comparison

Genome alignment was performed using NUCMER (Delcher, Phillippy, Carlton & Salzberg, 2002) to detect genomic inversions and other genomic differences. In addition, genomic rearrangements and inversions were visualized using Mauve (v 2.4.0) (Darling, Mau, & Perna, 2010).

### Calculation of Pan-Genome Orthologous Groups

Pan-genome orthologous groups (POGs) were identified by the combined reciprocal best hit (RBH) method using uBLAST with an e-value threshold of 1×10⁻⁶ (Ward & Moreno-Hagelsieb, 2014). In addition, an ORF-independent method using nucleotide sequences with cutoff values of at least 70% of gene coverage was applied (Chun et al., 2009). Some genes that were grouped due to short sequence length were further analyzed for clustering against the POGs using UCLUST (≥95% identity). A Venn diagram was created using jvenn to visually represent the calculated POGs (Bardou, Mariette, Escudie, Djemiel & Klopp, 2014).

Analysis of Genes Related with Health Benefits

Targeted genomic analysis was performed on the CAB701 strain and the WiKim0124 strain, focusing on genes associated with immune regulation and anti-obesity functions. The CDSs of both strains were scanned using bioinformatics tools. The focus was to identify genes associated with cell surface factors, such as lipoteichoic acid (LTA) and extracellular polysaccharides (EPS), and genes associated with short-chain fatty acids (SCFAs) and extracellular vesicles (EVs).

### Test Results

### 2-1: Genomic Characterization and Annotation

### 2-1-1: General Genomic Characteristics

The genomes of the CAB701 and WiKim0124 strains were analyzed and compared in Table 3 below.

TABLE 3

| | CAB701 | | WiKim0124 | |
|---|---|---|---|---|
| Assembly type | Complete | | Incomplete (contigs) | |
| GC content | 35.0% | | 34.8% | |
| Chromosome length | 2,575,822 | | 2,505,913 | |
| (genome size, bp) | | | | |
| Mean length of intergenic region | 141.4 | (169.6) | 148.4 | (206.3) |
| Mean of CDS length (s.d) (bp) | 911.5 | (747.1) | 886.1 | (611.7) |

| Median of CDS length (bp) | 759 | 763.5 |
|---|---|---|
| N50 (bp) | 2,517,318 | 2,505,913 |
| No. of coding sequences (CDSs) | 2,445 | 2,420 |
| No. of contigs | 2 | 1 |
| No. of rRNA genes | 19 | 19 |
| No. of tRNA genes | 63 | 70 |
| Sequencing depth of coverage | 448.01x | — |

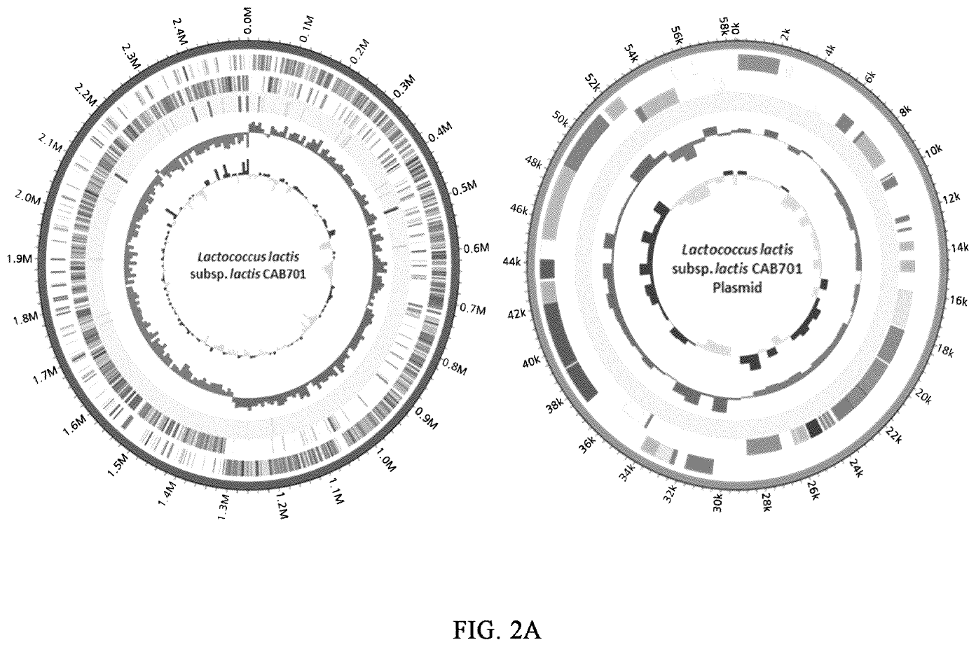

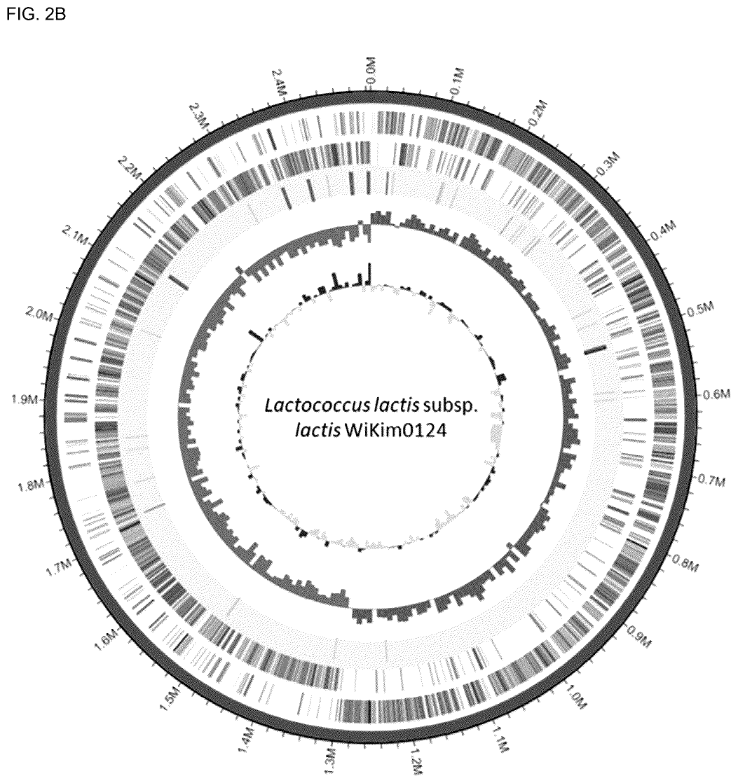

The complete genome sequence of the CAB701 strain consists of a circular chromosome and a plasmid (FIGS. 2A and 2B). Looking at Table 3, the chromosome of the CAB701 strain is 2,517,318 bp long with a GC content of 35.00%, and the plasmid is 58,504 bp long with a GC content of 34.09%. Furthermore, the genome encodes 2,445 CDSs along with 63 tRNA genes and 19 rRNA genes, providing a comprehensive genomic profile of this strain.

Meanwhile, the genome of L. lactis subsp. lactis WiKim0124 was analyzed based on the sequence obtained from the NCBI after uploading to the EzBioCloud database. The chromosome of the WiKim0124 strain is 2,505,913 bp long, has a GC content of 34.8%, and contains 2,420 CDSs. The WiKim0124 strain was also found to contain 19 rRNA genes and 70 tRNA genes, which was slightly more compared to L. lactis subsp. lactis CAB701.

### 2-1-2: CAZy Enzyme Annotation and Classification

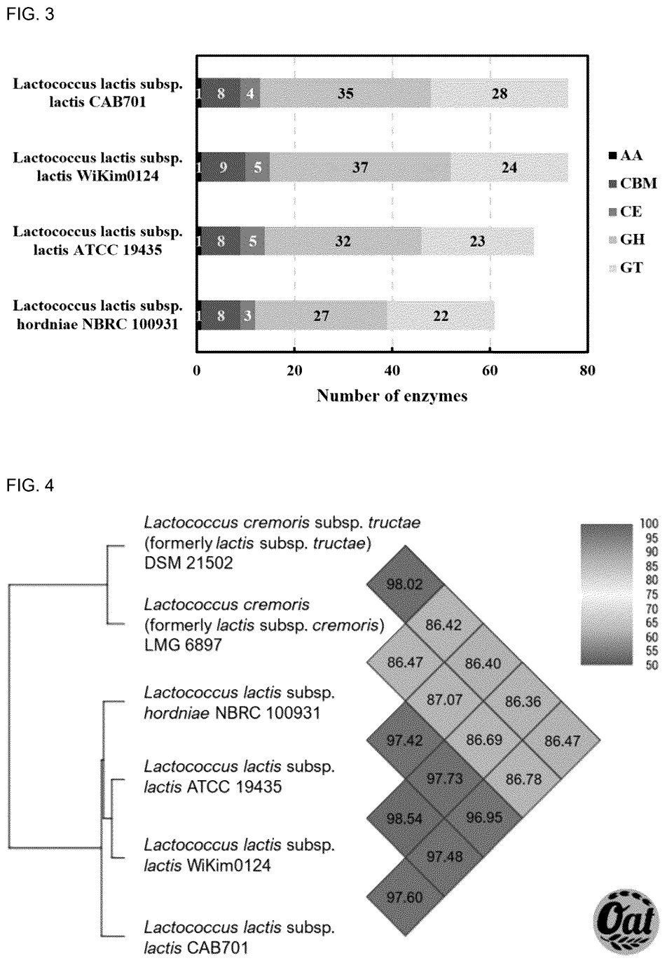

CAZyme annotation and classification of the L. lactis strains were performed using the dbCAN3 server, and the result is shown in FIG. 3. The core enzymes of the L. lactis strains were classified into five groups: accessory activity (AA), carbohydrate-binding module (CBM), carbohydrate esterase (CE), glycoside hydrolase (GH), and glycosyltransferase (GT).

Referring to FIG. 3, it can be seen that the CAB701 strain of the present disclosure has 28 GTs, 35 GHs, 4 CEs, 8 CBMs and 1 AA, indicating that it has the highest activity of glycosyltransferase. On the other hand, the WiKim0124 strain has 24 GTs, 37 GHs, 5 CEs, 9 CBMs, and 1 AA, indicating that it has enhanced carbohydrate processing capability. In addition, the L. lactis subsp. hordniae NBRC 100931 strain exhibited a profile with one AA, eight CBMs, three CEs, 27 GHs, and 22 GTs. The L. lactis subsp. lactis ATCC 19435 was found to have 1 AA, 8 CBMs, 5 CEs, 32 GHs, and 23 GTs, showing a slightly improved profile in CEs and GHs as compared to the NBRC 100931 strain.

### 2-2: Comparative Genomic Analysis

### 2-2-1: Phylogenetic Relationship

OrthoANI values were calculated to reveal the phylogenetic relationship between the L. lactis strains (FIG. 4).

Referring to FIG. 4, the CAB701 strain of the present disclosure and the WiKim0124 strain show a close genetic relationship with the OrthoANI value of 97.597%. The L. lactis subsp. lactis ATCC 19435 strain was found to have a very close genetic relationship with the CAB701 strain and the WiKim0124 strain, with high OrthoANI values of 97.48% and 98.54%, respectively, and was identified as L. lactis subsp. lactis.

### 2-2-2: Genome Alignment and Comparison

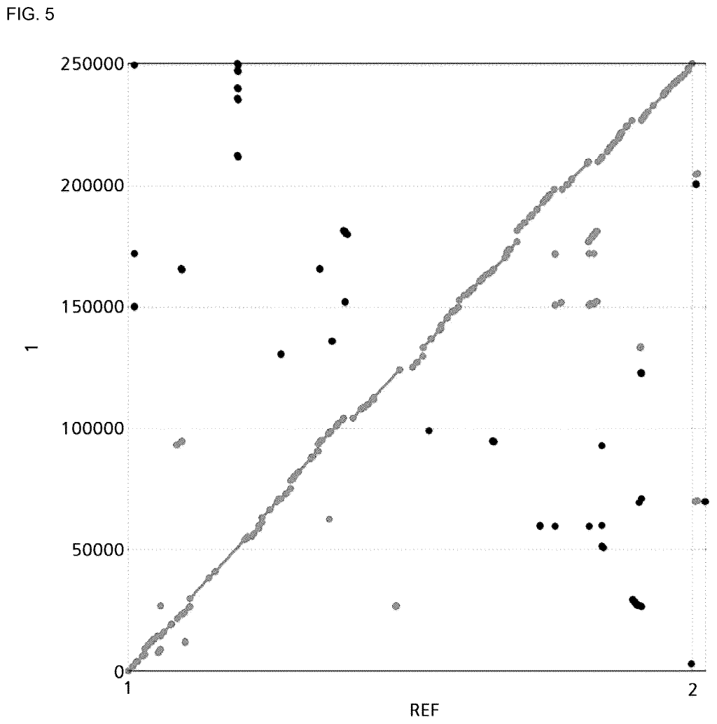

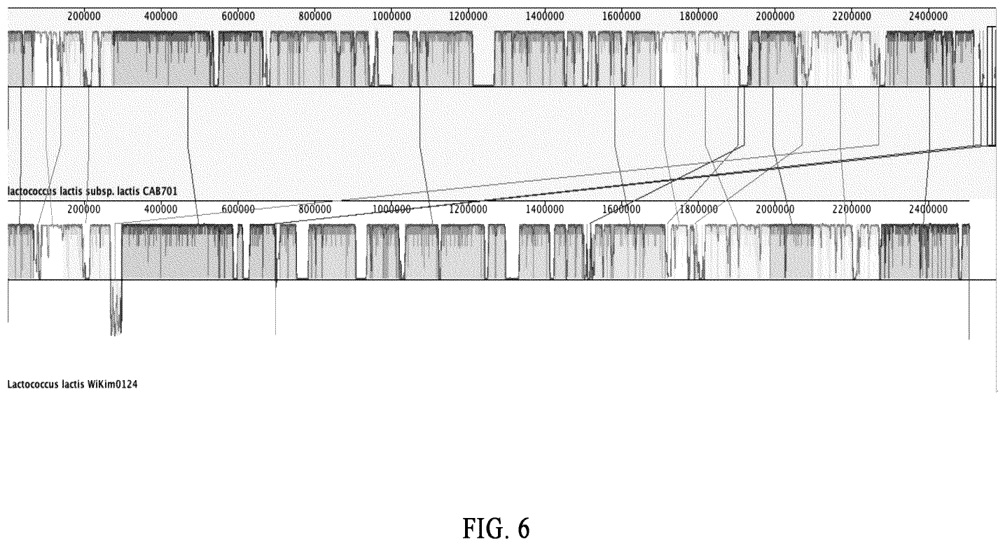

The genomes of the CAB701 strain of the present disclosure and the WiKim0124 strain were aligned and compared using NUCMER and Mauve (FIGS. 5 and 6).

As shown in FIG. 5, the NUCMER alignment indicates significant regions of homology between the genomes of the two L. lactis strains, as evidenced by the diagonal line. On the other hand, the deviation from the diagonal line, which appear as scattered dots, indicate genomic rearrangements, including insertion, deletion or inversions, and differences between the two strain genomes.

As shown in FIG. 6, the Mauve alignment provides insight into localized blocks of overlap between the genomes of the two L. lactis strains. In FIG. 6, the colored blocks represent regions of homologous DNA between the genomes of two L. lactis strains without sequence rearrangements, indicating a high degree of homology. Meanwhile, the gaps or misaligned regions in the colored blocks represent genomic differences between the two strains.

2-2-3: Pan-Genome Organization of Lactococcus lactis Strains

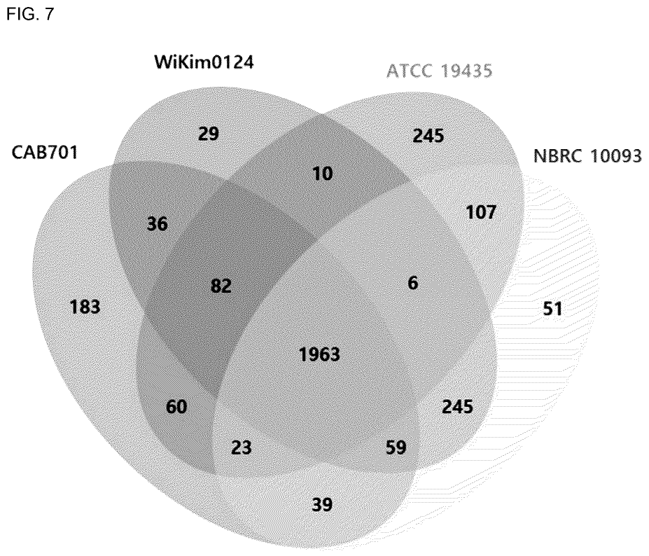

Through pan-genome analysis, 1,963 core genes shared by the four L. lactis strains CAB701, WiKim0124, ATCC 19435 and NBRC 10093, were identified (FIG. 7). Furthermore, as shown in FIG. 7, the CAB701 strain of the present disclosure was found to have 183 unique genes. These 183 unique genes may be related to unique metabolic pathways or survival mechanisms that are not present in other strains. It was also found that the WiKim0124 strain has 29 unique genes, the ATCC 19435 strain has 245 unique genes, and the NBRC 10093 strain has 51 unique genes.

2-3: Analysis of Genes Related with Health Benefits

As a result of analysis of genes related with health benefit, it was revealed that the CAB701 strain of the present disclosure uniquely possesses the IgA-specific serine endopeptidase gene, unlike other strains, indicating that the CAB701 strain may be specialized for immune regulation.

On the other hand, both the CAB701 strain and the WiKim0124 strain contain the diacylglycerol kinase (ATP), D-alanine poly(phosphoribitol) ligase, CDP-ribitol-ribitol phosphatase, teichoic acid poly(glycerol phosphate) polymerase, and teichoic acid translocation permease protein TagG genes, which all contribute to the composition of LTA and TA. Both strains were found to have genes for exopolysaccharide (EPS) biosynthesis. In addition, the two strains were detected to have genes involved in production of small peptides such as lactose and serine/threonine protein kinases. The CAB701 strain had a higher number of the serine/threonine protein kinases than the WiKim0124 strain. For the cell wall proteins, both strains were observed to have D-alanine-D-alanine ligase, D-aspartate ligase, and glutamate racemase genes in common. In addition, both strains were found to possess penicillin-binding protein and pethidoglycan synthase genes, which are important for cell wall construction and maintenance.

Furthermore, both strains showed the presence of genes involved in short-chain fatty acid (SCFA) biosynthesis, specifically the acetic acid kinase gene. In addition, both strains had four copies of the L-lactate dehydrogenase gene, indicating the ability to utilize and convert lactate. In addition, the genes encoding formate C-acetyltransferase and [formate C-acetyltransferase] activator were also identified in equal numbers in both strains.

### 2-4: Sub-Conclusion

In the gene function annotation study of the CAB701 strain of the present disclosure, recurrent annotations for certain enzymes, particularly GH1 and GT2, indicate that they play an important role in the organism's carbohydrate metabolism. Specifically, the CAB701 strain has 9 annotations related to the GH1 enzyme, which is known for its ability to hydrolyze the β-glycosidic bonds of various carbohydrates such as lactose, cellobiose and certain glycosides, suggesting that it can efficiently process carbohydrates. In addition, the 15 GT2 enzyme-related annotations found to be present are essential for the formation of glycosidic bonds, which are important for synthesizing cell wall components, extracellular polysaccharides or glycoproteins, suggesting that this biosynthetic capacity may elaborate carbohydrate metabolism.

In conclusion, it is expected that the CAB701 strain of the present disclosure with higher GH and GT activities has a relatively high fermentation efficiency for complex carbohydrates, suggesting that it may make be a good candidate for probiotics to aid in the digestion of dairy products or dietary fibers.

### Example 3: Optimization of Culturing

### Experimental Methods

### Preparation and Fermentation Condition of Culture

The CAB701 strain of the present disclosure was prepared by subculturing for at least two passages on MRS agar before preparing an inoculum. Then, the single colony of the CAB701 strain was cultured in MRS medium for 16 hours at 37° C. before being used as an inoculum. To optimize initial fermentation conditions such as the medium, temperature and pH, 1% (v/v) inoculum was added to 30 mL of MRS medium and incubated for 10 hours at 37° C. Monitoring was preformed using RTS-8 Plus (Biosan, Riga, Latvia).

### Setting of Carbon Source Concentration

0-5% (w/v) sucrose was added to the optimized medium. The optical density at 600 nm was then monitored to create a growth curve, and the number of viable cells was determined at 6 and 10 hours after the inoculation to assess the effect of the sucrose concentration on bacterial proliferation.

### Validation of Anti-Obesity Efficacy of CAB701 Strain in Optimized Medium

Lipid accumulation in 3T3-L1 preadipocytes was analyzed using the Oil Red O staining method. 3T3-L1 adipocytes obtained from the ATCC (CL-173™) were cultured in Dulbecco's modified Eagle's medium (DMEM) supplemented with newborn calf serum and antibiotics. The cells were maintained at 37° C. and 5% CO₂. For differentiation, the cells were seeded into a 6-well plate, grown to confluence, and exposed to a differentiation medium containing 3-isobutyl-1-methylxanthine, dexamethasone and insulin, followed by maintenance in an insulin-supplemented differentiation medium. The CAB701 strain was then cultured at a density of 1×10⁸ CFU/mL in Lactobacilli de Man, Rogosa, and Sharpe medium. The centrifuged and washed CAB701 strain was then introduced to the 3T3-L1 cells. A control group consisted of cells exposed only to the differentiation medium or maintained in a growth medium without the CAB701 strain. After differentiation, the 3T3-L1 cells were subjected to Oil Red O staining according to an established protocol. The stained cells were observed under a microscope and the dye extracted with isopropanol was measured quantitatively at 500 nm.

### Test Results

### 3-1: Optimization of Medium

### 3-1-1: Effect of Carbon and Nitrogen Sources on Growth of CAB701 Strain

It was found that the optimization of the fermentation medium using the OFAT method had a significant impact on the growth of the CAB701 strain.

Specifically, when various carbon sources were added at 20 g/L, sucrose and maltose were the most effective, producing the highest number of living cells of 9.44 log CFU/mL and 9.39 log CFU/mL, respectively, after 10 hours of culturing. Similarly, when various nitrogen sources were added at 25 g/L, the yeast extract was found to be the most conducive to cell proliferation, with the final number of living cells of 9.51 log CFU/mL in the yeast extract addition group after 10 hours of culturing. In contrast, the isolated soy protein addition group showed relatively low cell viability, with the final cell count of 8.54 log CFU/mL. These results suggest that the type of carbon and nitrogen sources in the growth medium has an important influence on the growth of the CAB701 strain.

### 3-1-2: Identification of Growth Factors of CAB701 Strain

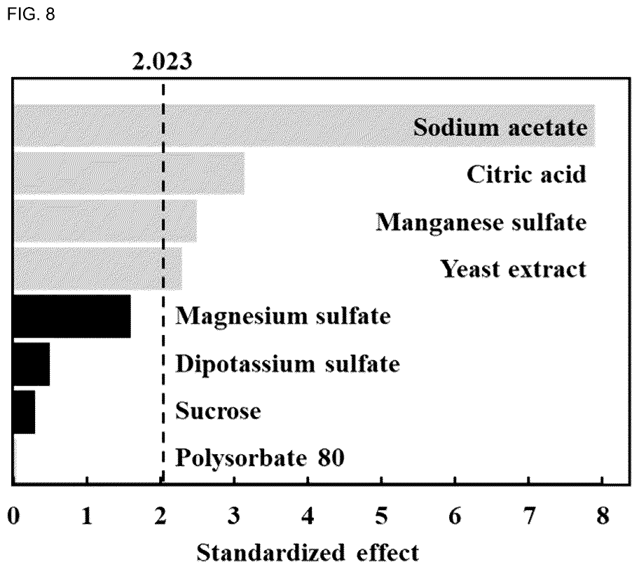

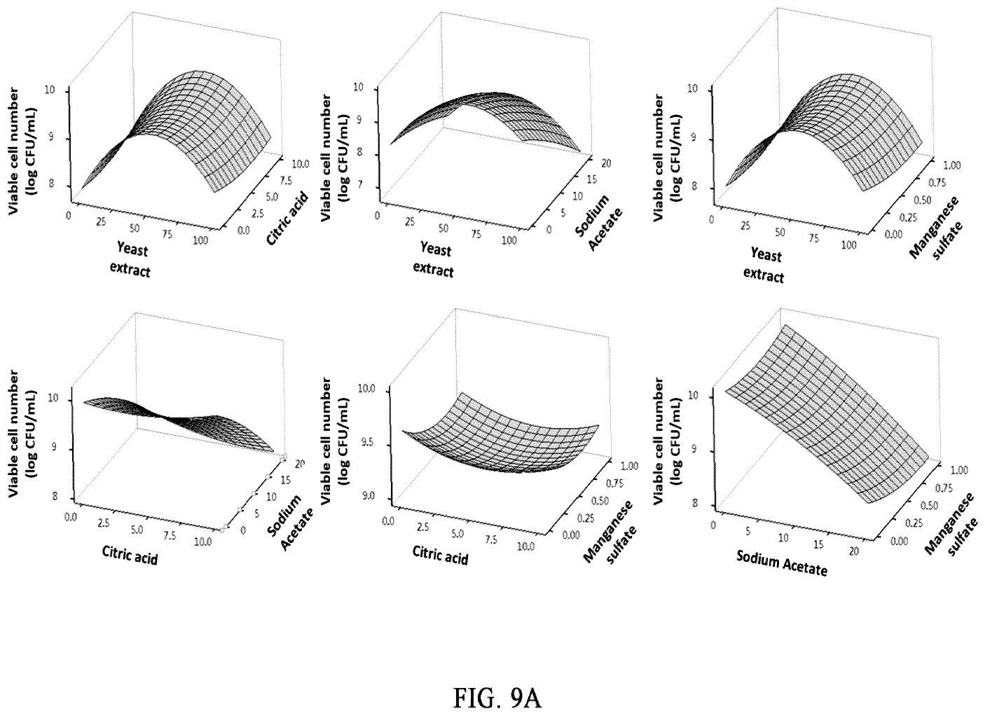

The effect of multiple factors on the growth of the CAB701 strain were evaluated using the Plackett-Burman design (FIG. 8). The effects of variables from the carbon source to inorganic salts were adjusted at two levels, low (−1) and high (+1), as shown in Table 4 below. 48 experiments were conducted in total, with each condition repeated twice to ensure the reliability of the results.

TABLE 4

| | Independent | Variable | | |
|---|---|---|---|---|
| Source | variables | code | Low (−1) | High (+1) |
| Carbon | Sucrose | X₁ | 1 | 100 |
| source | | | | |
| Nitrogen | Yeast extract | X₂ | 5 | 100 |
| source | | | | |
| Others | Polysorbate 80 | X₃ | 0.1 | 2 |
| | Citric acid | X₄ | 0.5 | 10 |
| | Sodium acetate | X₅ | 1 | 20 |
| | Magnesium sulfate | X₆ | 0.1 | 2 |
| | Manganese sulfate | X₇ | 0.01 | 1 |
| | Potassium | X₈ | 0.5 | 10 |
| | phosphate | | | |

As shown in FIG. 8, four components were identified to have standardized effects exceeding the threshold of 2.023. Those components were sodium acetate, citric acid, manganese sulfate and yeast extract, and were found to have the greatest impact on the number of living cells (log CFU/mL) in the bacterial culture.

### 3-1-3: Optimization of Medium Ingredients

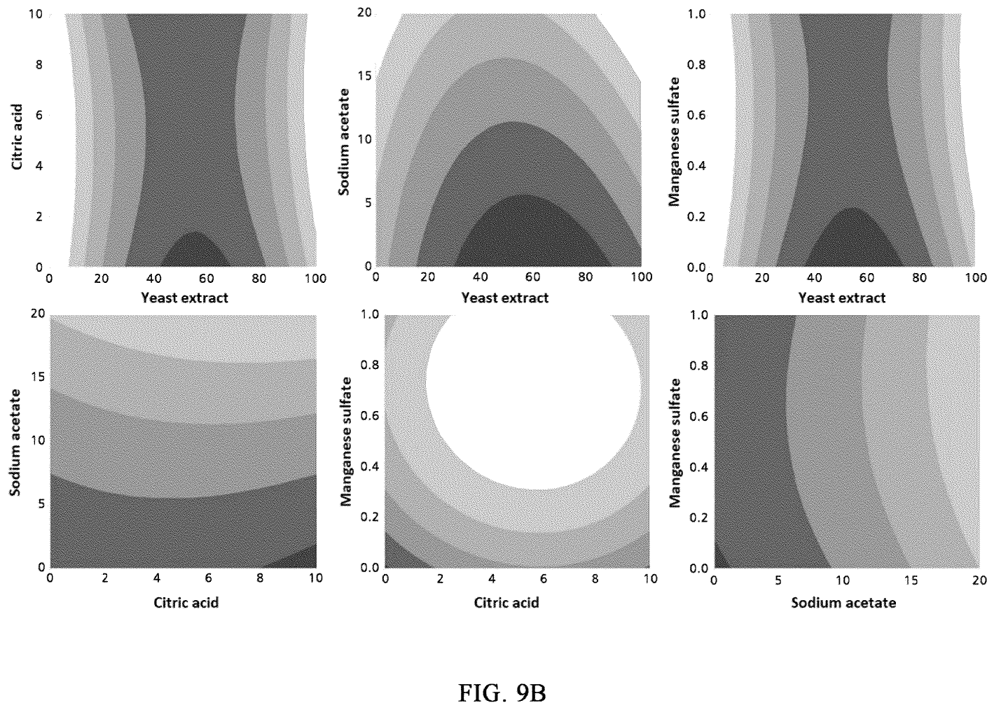

The medium ingredients (yeast extract, citric acid, sodium acetate, and manganese sulfate) for the CAB701 strain were optimized using the response surface methodology (RSM) and artificial neural network (ANN). Specific experiments were run 90 times, including 30 base experimental runs repeated 3 times. The results were presented as three-dimensional and contour plots of the RSM showing the combined effect of the four variables on the number of viable cells of the strain (FIGS. 9A and 9B). Looking at FIGS. 9A and 9B, it can be seen that the optimal medium composition is 60 g/L (6%) of yeast extract and 10 g/L (1%) of citric acid, with no sodium acetate and/or manganese sulfate added. The above medium composition provided a predicted number of living cells of 10.37 log CFU/mL, which closely matched the experimental value of 9.72 log CFU/mL with a moderate MSE of 0.419.

### 3-1-4: Effect of Sucrose Concentration on Strain Growth

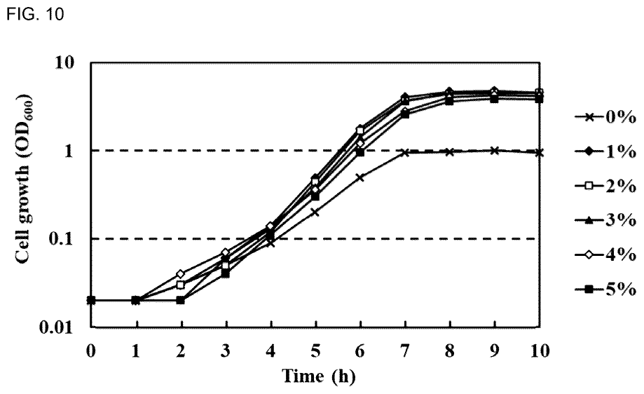

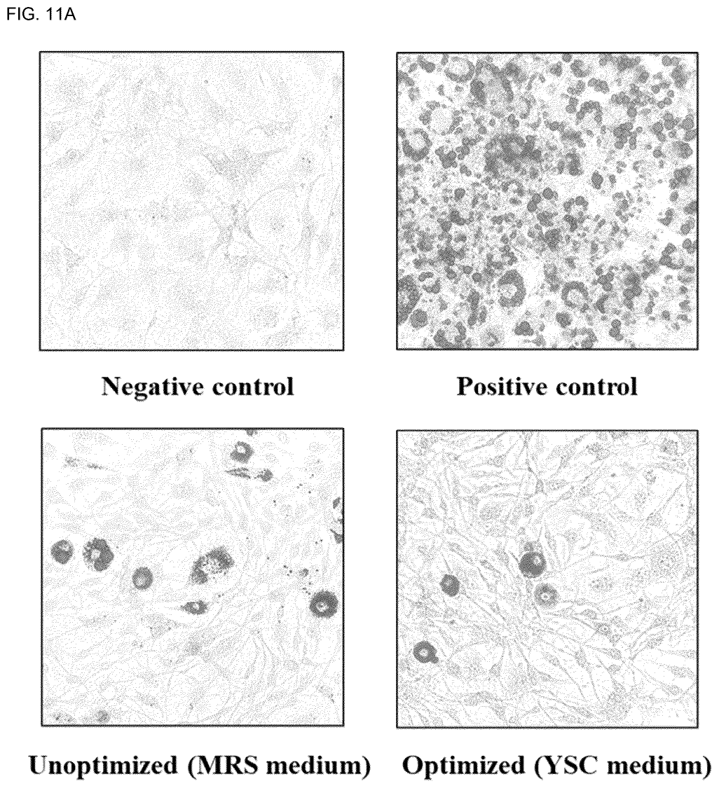

The growth of the CAB701 strain was observed. In the absence of sucrose, the growth of the CAB701 strain was reduced significantly, with the number of living cells reaching 8.95 log CFU/mL. In contrast, the inclusion of sucrose significantly increased the proliferation of the strain (FIG. 10).

Looking at FIG. 10, it can be seen that the number of living cells is relatively consistent at various sucrose concentrations, with 9.87 log CFU/mL at 1% sucrose concentration, 9.58 log CFU/mL at 2%, 9.87 log CFU/mL at 3%, 9.79 log CFU/mL at 4%, and 9.69 log CFU/mL at 5%.

### 3-1-5: Validation of Anti-Obesity Efficacy of CAB701 Strain in Optimized Medium

In Test Examples 3-1-3 and 3-1-4 described above, the optimal medium composition for the CAB701 strain was found to be MRS medium (YSC medium) containing 1% (w/v) sucrose, 6% (w/v) yeast extract, and 1% (w/v) citric acid.

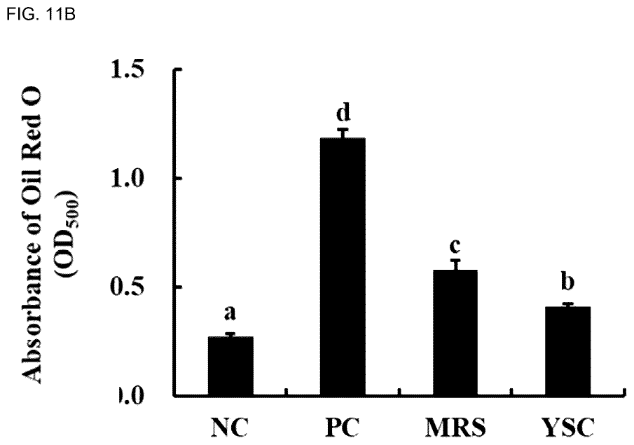

The anti-obesity activity of the strain was validated by measuring lipid accumulation in 3T3-L1 preadipocytes treated with the CAB701 strain using the Oil Red O staining method described in Test Example 6-3 (FIGS. 11A and 11B).

As shown in FIGS. 11A and 11B, the untreated negative control group showed an absorbance of 0.272, indicating negligible lipid accumulation with a differentiation rate of 0%, while the fully differentiated positive control group showed an absorbance of 1.189, corresponding to a differentiation rate of 100%. Furthermore, the 3T3-L1 preadipocytes exposed to the CAB701 strain cultured in a non-optimized MRS medium exhibited an absorbance of 0.579 and a differentiation rate of 33.52%. In contrast, the 3T3-L1 preadipocytes exposed to the CAB701 strain cultured in the optimized medium (YSC medium) showed significantly reduced absorbance differentiation rate of 0.411 and 15.14%, respectively.

### 3-2: Optimization of Culturing Condition

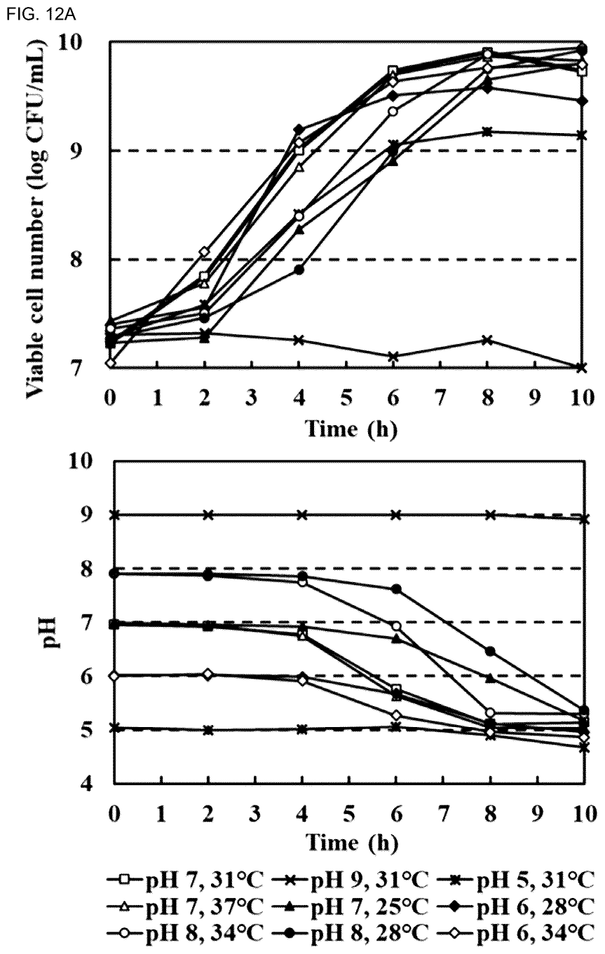

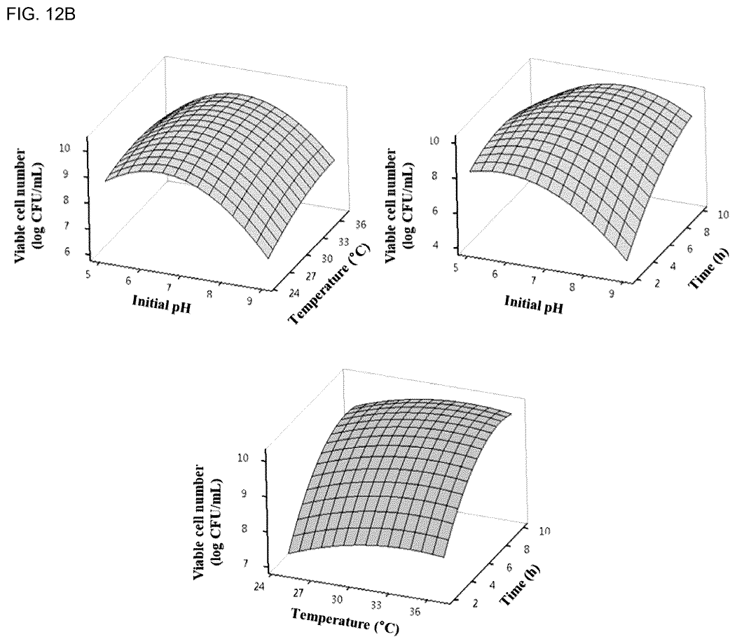

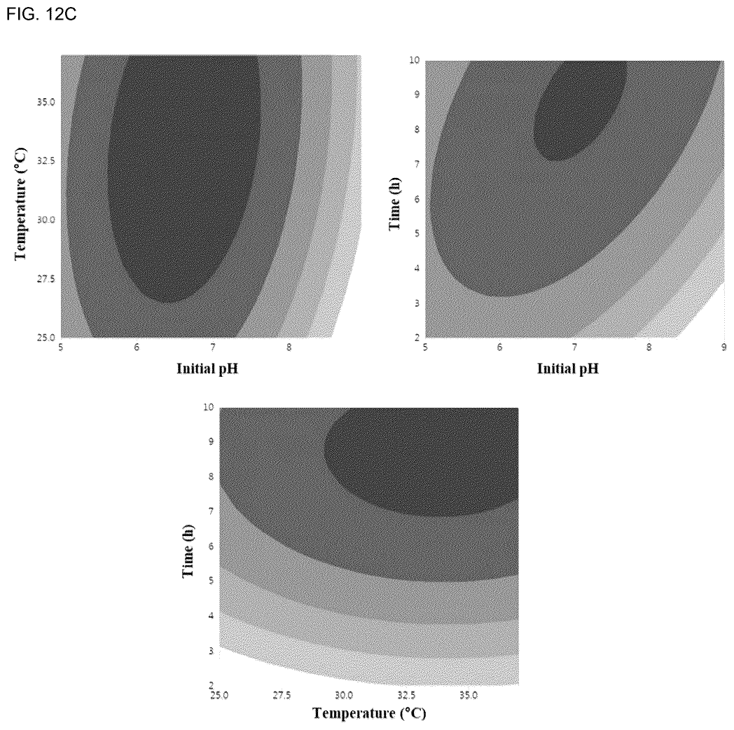

The condition for culturing the CAB701 strain (initial pH, temperature and time) was optimized using the response surface methodology (RSM) and artificial neural network (ANN). Specific experiments were run 20 times for each method. The result was presented as three-dimensional and contour plots of the RSM showing the combined effect on the number of viable cells of the strain (FIGS. 12A to 12C).

Looking at FIGS. 12A to 12C, the optimal fermentation condition was pH 7.37, 34.4° C. and 8.69 hours. The predicted cell count was 9.94 log CFU/mL, which was in perfect agreement with the experimental value of 9.92 log CFU/mL (MSE 0.00).

### 3-3: Sub-Conclusion

In conclusion, the optimal medium composition for the CAB701 strain of the present disclosure, which was optimized using RSM and ANN, was found to be a medium containing 1% (w/v) sucrose, 6% (w/v) yeast extract, and 1% (w/v) citric acid. It has been shown that culturing of the CAB701 strain of the present disclosure in the optimized medium further enhances strain growth and significantly improves the anti-obesity activity of the strain as compared to culturing in the conventional MRS medium (commercial MRS medium). The optimal culturing condition for the CAB701 strain of the present disclosure was found to be an initial pH of 7.37 and a temperature of 34.4° C. The culturing condition is essential for mass production of the strain, is economical, and increases probiotic yields.

### <Functional Evaluation In Vitro>

### Example 4: Antioxidant Activity-DPPH Radical Scavenging Activity

The antioxidant activity of the CAB701 strain was measured by DPPH (2,2-diphenyl-1-picrylhydrazyl, Sigma-Aldrich, St. Louis, MO, USA) radical scavenging assay. Standard curves were created using 0.0625, 0.125, 0.25, 0.5, 1 and 2 mM ascorbic acid. 2 mM ascorbic acid was used as a positive control group, and tertiary distilled water used as a negative control group.

The culture of the CAB701 strain cultured in MRS broth at 37° C. for 20 hours was inoculated to MRS broth at 1% (v/v) and incubated at 37° C. for 16 hours. After centrifuging the culture of the CAB701 strain at 17,000 rpm for 5 minutes, 50 L of the culture supernatant or control group was transferred to a 1.5-mL tube. 0.1 mM DPPH diluted in methanol was used in the experiment. 950 μL of 0.1 mM DPPH was added to a 1.5-mL tube containing the CAB701 strain culture supernatant and incubated in the dark at room temperature for 30 minutes. The reaction mixture was centrifuged at 13,000 rpm for 1 minute. 200 μL of the supernatant was dispensed into a 96-well plate, and absorbance was measured at 517 nm using a microplate reader (Epoch microplate reader, Biotek Instruments, Inc., Winooski, VT, USA). DPPH radical scavenging activity was calculated using the following equation and expressed as a relative value to the DPPH radical scavenging activity of the positive control group, 2 mM ascorbic acid. The result is shown in Table 5 below.

DPPH Radical Scavenging Activity (%)=(1-(Absorbance of Sample/Absorbance of Control Group))×100

TABLE 5

| | DPPH radical scavenging activity (%) |
|---|---|
| CAB701 | 95.6 ± 1.3 |
| Lactobacillus rhamnosus GG | 97.2 ± 0.6 |

Referring to Table 5, it can be seen that the comparative strain Lactobacillus rhamnosus GG (LGG) has a DPPH radical scavenging activity of 97.2%, and the CAB701 strain isolated in the example has a DPPH radical scavenging activity of 95.6%, both of which are higher than 90%.

### Example 5: Immune-Enhancing Activity

### Experimental Methods

### Cell Line and Culturing Condition Thereof

RAW 264.7 cells (KCLB40071), which are mouse macrophages, were purchased from the Korean Cell Line Bank. They were inoculated to DMEM (high-glucose Dulbecco's modified Eagle's medium; GIBCO, Grand Island, NY, USA) containing 10% (v/v) fetal bovine serum (FBS, GIBCO) and 1% (v/v) penicillin-streptomycin (P/S, GIBCO), cultured at 37° C. in a 5% CO₂ incubator (Thermo Fisher Scientific, Waltham, MA, USA), and then detached using a cell scraper (SPL, Pocheon, Korea).

### Preparation of Strain Sample

The CAB701 strain was cultured in MRS broth for 16 hours at 37° C., and inoculated to 1% in 10 mL of MRS broth to a concentration of 1×10⁸ CFU/mL at 37° C. Cell precipitates were harvested by centrifugation at 10,000 rpm for 5 minutes at 4° C. for 5 times. Afterward, they were washed three times with DPBS (Dulbecco's phosphate buffered saline; Welgene, Gyeongsan, Korea). These were resuspended in DMEM supplemented with 10% (v/v) FBS and 1% (v/v) P/S and prepared by diluting the samples to the desired MOI (multiplicity of infection; viable Lactobacillus cell count/RAW264.7 cell count) of the CAB701 strain relative to the macrophage count.

### Test Results

### 5-1: Cytotoxicity of Strain

Before the measurement of the immunomodulatory capacity of the CAB701 strain, the cytotoxicity against RAW 264.7 was determined.

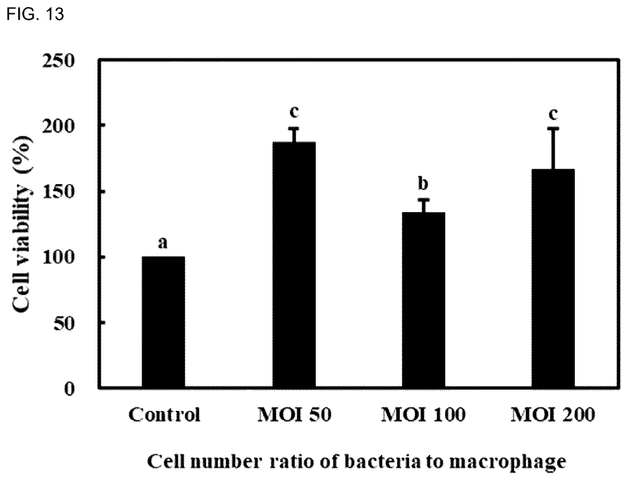

First, 100 μL of RAW 264.7 cells were seeded in a 96-well plate at a density of 5×10⁴ cells/well and incubated at 37° C. and 5% CO₂ for 20 hours. MOI 0 was used as a control group, and 100 μL of each of samples of the CAB701 strain at MOI 50, 100 and 200 was incubated at 37° C., 5% CO₂ for 24 hours. Cytotoxicity was measured using an EZ-CYTOX kit (Daeillab, Korea). After the incubation, the medium was removed and 200 μL of DMEM supplemented with 10% FBS and 1% P/S and 20 μL of Ez-Cytox were added to each well, followed by reaction at 37° C. in a 5% CO₂ incubator for 30 minutes. As a blank, a well without cells was treated in the same way. To measure the viability of the RAW264.7 cells, absorbance was measured at 450 nm using a microplate reader when the medium turned orange, and the cell viability was calculated using the following equation (FIG. 13). The absorbance of the DMEM medium and Ez-Cytox alone added to a well without cell seeding was used as the blank value.

$$Cell viability ( \% ) = [ ( test group - blank ) / ( control group + blank ) ] \times 100$$

From FIG. 13, it can be seen that the CAB701 strain showed no cytotoxicity at concentrations below MOI 200, and the cell viability exceeded 150% even when treated with a concentration of MOI 200.

### 5-2: Facilitation of NO Production

NO is produced by macrophages or neutrophils, and an adequate amount of NO secreted by activated immune cells acts as an immune signaling molecule, thereby activating immune cells to provide protection against foreign pathogens. By measuring the amount of NO produced by cytokines and endotoxins such as LPS, the immune activity of the CAB701 strain against was compared with that of the L. rhamnosus GG (LGG) strain.

NO production by RAW 264.7 macrophages was measured using a Griess reagent. 500 μL of RAW 264.7 cells were seeded in a 96-well plate at a density of 5×10⁵ cells/mL and incubated at 37° C. and 5% CO₂ for 20 hours. The RAW 264.7 cells were treated with each of the CAB701 strain (5×10⁷ CFU) corresponding to MOI 100, a positive control group LPS (1 μg/mL) and a negative control MOI 0, and then cultured for 24 hours. 100 UL of the culture was added to a 96-well plate and, after adding 100 μL of a Griess reagent A and a Griess reagent B of equal amounts, reaction was conducted for 15 minutes in the dark, and absorbance was measured at 540 nm with a microplate reader. A standard curve for NO production versus absorbance was constructed using a sample of 250 UM NaNO₂ diluted 2-fold with DMEM to 7.815 UM, and NO concentration was calculated from the measured absorbance value. The NO production in the sample treated with the CAB701 strain, with respect to the NO production of the positive control group treated with 1 μg/mL of LPS as 100%, is shown in Table 6 below.

TABLE 6

| | NO production |
|---|---|
| | (% of positive control group) |
| CAB701 | 86.3 ± 6.8 |
| Lactobacillus rhamnosus GG | 18.5 ± 3.2 |

Looking at Table 6, it can be seen that the CAB701 strain produced 86.32% of NO as compared to LPS, which was significantly higher than the NO production by the comparative strain LGG (18.47%). From the above result, it can be seen that the CAB701 strain has good immune-enhancing activity.

### 5-3: Increased Expression of Cytokine-Related Genes

The immune activity mechanism of the CAB701 strain was investigated by treating RAW 264.7 cells with the CAB701 strain, which showed the highest NO production in the NO assay, and then identifying the relative mRNA expression level of genes regulating cytokine production by real-time quantitative PCR (RT qPCR).

RAW 264.7 cells were seeded in a 6-well plate at a density of 1×10⁶ cells/well and incubated at 37° C., 5% CO₂ for 20 hours. The RAW 264.7 cells were treated with the CAB701 strain at an MOI of 100, a positive control group was treated with 1 μg/mL LPS, and a negative control group was treated with MOI 0, for 24 hours at 37° C. and 5% CO₂. After the incubation, the cells were washed twice with DPBS. The cells were isolated using 1 mL of an easy-BLUE™ total RNA extraction kit (iNtRON Biotechnology, Inc., Seongnam, Korea) and collected in a 1.5-mL tube. 200 μL of chloroform (Sigma-Aldrich) was added and mixed by vortexing for 10 seconds. After centrifugation at 13,000 rpm for 10 minutes at 4° C., 400 μL of the supernatant was transferred to a new 1.5-mL tube. 400 μL of 2-propanol (Sigma-Aldrich) was added, mixed, and allowed to react for 10 minutes at room temperature. After centrifugation at 13,000 rpm for 5 minutes at 4° C., the supernatant was removed, and the RNA pellet was washed with 1 mL of 75% ethanol (Sigma-Aldrich) and centrifuged at 10,000 rpm for 5 minutes at 4° C. After drying the RNA pellet as much as possible, the RNA pellet was dissolved in 30 μL of DEPC-DW for RNA (Bioneer, Daejeon, Korea).

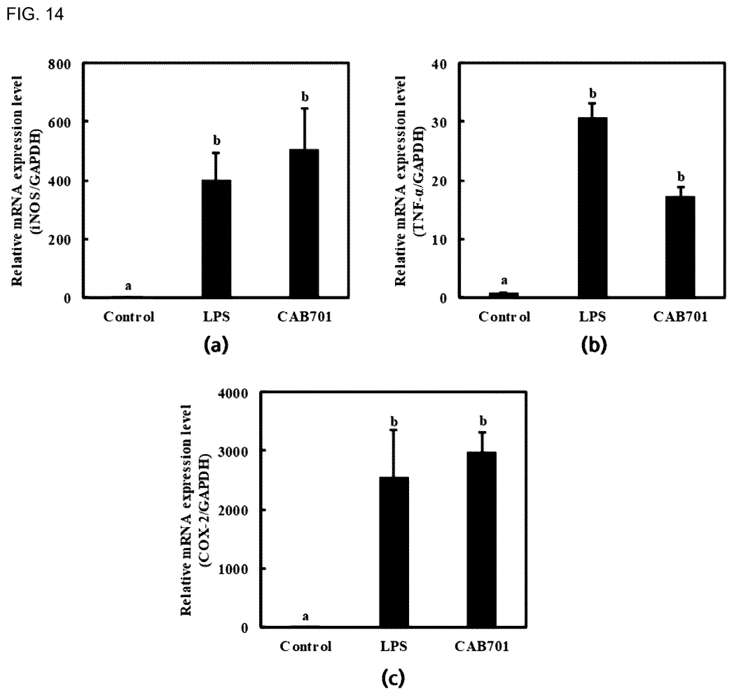

After RNA extraction, the concentration and purity of the extracted RNA was measured for cDNA synthesis using a Take3 micro-volume plate (Epoch microplate reader, Biotek Instruments, Inc.). A CellScript™ cDNA master mix (Cellsafe, Suwon, Korea) was used to synthesize cDNA on a PCR machine. The reaction master mix contained an oligo (dT) s primer, a MIMLV reverse transcriptase (Rlase), dNTPs, and a ribonuclease inhibitor at an optimized ratio. The PCR condition for cDNA synthesis is shown in Table 8. RT-qPCR was performed using the QuantStudio1 real-time PCR system (Thermo Fisher Scientific, Milan, Italy) using GreenBlue™ 2X Green qPCR Master Mix (Cellsafe) by adding 500 ng of cDNA and primers according to the manufacturer's instructions. Primer sequences are shown in Table 7, and RT-qPCR was performed according to Table 8. After normalization using the glyceraldehyde 3-phosphate dehydrogenase (GAPDH) gene as a housekeeping gene, the relative expression level of cytokine mRNA was calculated from the AA CT value. The result is shown in FIG. 14.

TABLE 7

| | | | | Size |
|---|---|---|---|---|
| No. | Gene | | Primer sequence | (bp) |
| 1 | GAPDH | F | 5′-CAT GGC CTT CCG | 122 |
| | | | TGT TCC TAC-3′ | |
| | | R | 5′-TCA GTG GGC CCT | |
| | | | CAG ATG C-3′ | |
| 2 | COX-2 | F | 5′-CTC AGC CAT ACA | 101 |
| | | | GCA AAT CCT T-3′ | |
| | | R | 5′-GTC CGG GTA CAA | |
| | | | TCG CAC TTA T-3′ | |
| 3 | iNOS | F | 5′-CCA GCC TGC CCC | 104 |
| | | | TTC AAT-3′ | |
| | | R | 5′-ATC CTT CGG CCC | |
| | | | ACT TCC T-3′ | |
| 4 | IL-1β | F | 5′-TGA CGG ACC CCA | 122 |
| | | | AAA GAT-3′ | |
| | | R | 5′-GTG ATA CTG CCT | |
| | | | GCC TGA AG-3′ | |
| 5 | IL-6 | F | 5′-CCG GAG AGG AGA | 107 |
| | | | CTT CAC AGA G-3′ | |
| | | R | 5′-TCA TTT CCA CGA | |
| | | | TTT CCC AGA G-3′ | |
| 6 | TNF-α | F | 5′-AGG CAC TCC CCC | 122 |
| | | | AAA AGA TG-3′ | |
| | | R | 5′-CAC CCC GAA GTT | |
| | | | CAG TAG ACA GA-3′ | |

TABLE 8

| | | Temperature | | | Number |
|---|---|---|---|---|---|
| Step | | (° C.) | Time | | of cycles |
| Hold stage | | 50 | 2 | min | 1 |
| | | 95 | 2 | min | |
| PCR stage | Denaturation | 95 | 15 | sec | 39 |
| | Annealing | 60 | 30 | sec | |
| | Extension | 72 | 15 | sec | 1 |

| Melting curve stage | 95 | 15 | sec |
|---|---|---|---|
| | 65 | 1 | min |
| | 97 | 1 | sec |

Referring to FIG. 14, the CAB701 strain of the present disclosure was shown to have excellent immune-enhancing activity, since it increases the expression of iNOS, COX-2 and TNF-α, which are cytokine-related genes, similarly to LPS.

### 5-4: Increased Expression of MAPKs Signaling Pathway-Related Proteins

To determine the mechanism of immune activity of the CAB701 strain, which showed high NO production in the NO assay, RAW 264.7 cells were treated with the CAB701 strain and the expression level of proteins related to the MAPKs signaling pathway was analyzed by western blot.

RAW 264.7 cells were treated with the CAB701 strain, washed twice with DPBS (Welgene), and reacted with 130 μL of a RIPA buffer (Cell Signaling Technology, Beverly, MA, USA) solution containing 1% (v/v) protease inhibition cocktail (GenDEPOT, Katy, TX, USA) and 1% (v/v) phosphatase inhibition cocktail (GenDEPOT) for 30 minutes at 0° C. using the same method as in the RNA isolation for RT-qPCR. The cells in the well were separated with a cell scraper, collected in a 1.5-mL tube, vortexed for 10 seconds, and reacted at 0° C. for 5 seconds. This process was repeated three times. After reaction at 0° C. for 2 hours, the cell lysate was centrifuged at 17,000 rpm for 10 minutes at 4° C., and the supernatant was transferred to a new 1.5-mL tube for protein extraction. The concentration of the extracted protein was quantified using a Pierce™ BCA protein assay kit (Thermo Fisher Scientific).

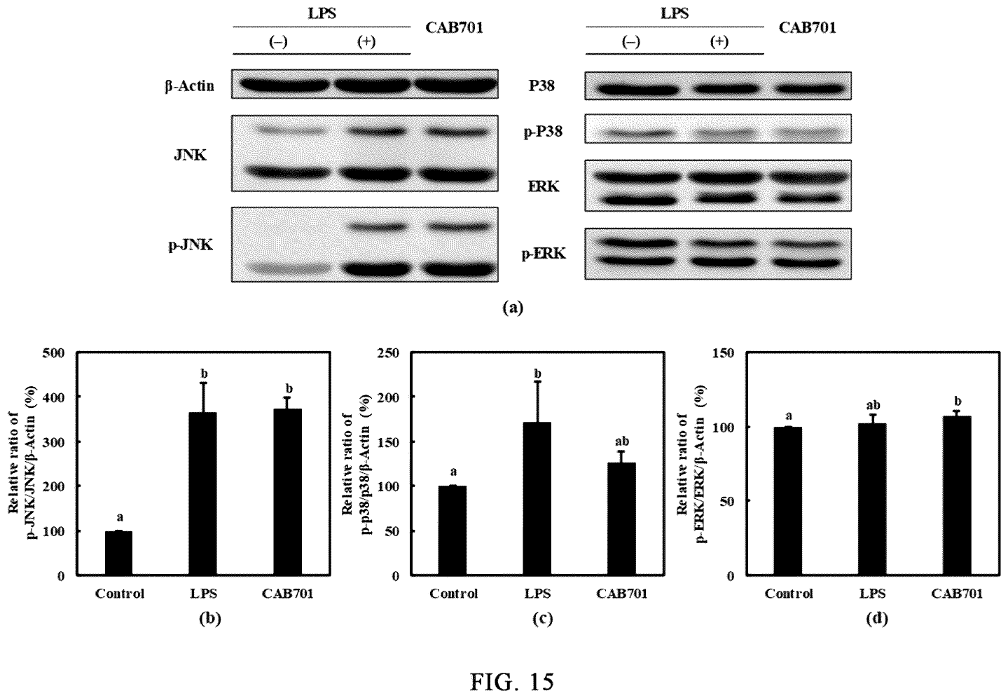

30 μg of the extracted protein was separated by 12.5% SDS-PAGE. For western blotting, the separated protein was transferred to a nitrocellulose membrane (Bio-Rad Laboratories, Inc.) using a TE70X semi-dry transfer unit (Hoefer Inc.) at 45 mV for 2 hours. 3% (w/v) bovine serum albumin fraction V (BSA, Roche) diluted with Tris-buffered saline (TBS-T, iNtRON Biotechnology) containing 0.5% (v/v) Tween 20 at room temperature was used as a blocking buffer. The transferred membrane was blocked for 30 minutes with gentle vortexing. The membrane was incubated with primary antibodies diluted to 1:1000 in 3% BSA in TBS-T for 1 hour at room temperature with gentle agitation. As the primary antibodies, p38, phospho-p38, ERK, phospho-ERK, JNK, phospho-JNK and β-actin (Cell Signaling Technology) were used. After washing four times with 6 mL of TBS-T every 15 minutes, the membrane was incubated with secondary antibodies diluted to 1:1000 in 3% BSA in TBS-T for 1 hour at room temperature with gentle agitation. Anti-rabbit IgG (Cell Signaling Technology) was used as the secondary antibody. After the reaction with the secondary antibodies, the reaction mixture was washed four times with 6 mL of TBS-T every 15 minutes, and then reacted with ECL solution (EzWestLumi plus, ATTO Corporation, Tokyo, Japan) and visualized using an Amersham™ M imager 600 (Cytiva, Amersham, England). Protein bands were quantified using the image J software (National Institutes of Health, Bethesda, MD, USA). β-Actin was used as a housekeeping protein for western blotting assay. Relative expression level was calculated with respect to the protein expression of the negative control group as 100 (FIG. 15).

Referring to FIG. 15, it can be seen that the macrophages treated with the CAB701 strain of the present disclosure exhibit a significant increase in the expression of MAPKs signaling pathway-related proteins, p-JNK/JNK (372%), p-p38/p38 (126%) and p-ERK/ERK (107%), as compared to the negative control group.

### Example 6: Anti-Obesity Activity

### 6-1: Inhibition of Pancreatic Lipase

To evaluate the body fat-reducing ability of the CAB701 strain according to the present disclosure, the inhibition of pancreatic lipase was examined.

50 mg of porcine pancreatic lipase type II (Sigma) was suspended in 10 mL of 10 mM Tris-HCl buffer adjusted to pH 8.0, vortexed for 15 minutes, and centrifuged at 4000 rpm for 10 minutes at 18° C. The supernatant was used as an enzyme solution. As a substrate, p-nitrophenyl butyrate (Sigma-Aldrich (Schnelldorf, Germany)) dissolved in acetonitrile to 10 mM was used.

A CAB701 strain culture incubated in MRS broth for 20 hours at 37° C. was centrifuged at 17,000 rpm for 5 minutes to obtain culture supernatant. Orlistat (Sigma-Aldrich), used as a positive control group, was diluted in DMSO to concentrations of 1000, 500 and 250 μg/mL. Tertiary distilled water was used as a negative control group. The final reaction mixture consisting of 880 μL of pH 8.0 10 mM Tris-HCl buffer, 100 UL of the enzyme solution, and 10 μL of the CAB701 strain culture supernatant or the positive control group was pre-incubated at 37° C. for 5 minutes. After adding 10 μL of a substrate solution, followed by reaction for 5 minutes at 37° C., absorbance was measured using a microplate reader at 405 nm, which is the maximum absorbance of p-nitrophenol. Pancreatic lipase inhibition rate was calculated using the following equation and the result is shown in Table 9 below.

Pancreatic lipase inhibition rate (%)=[(negative control absorbance-sample absorbance)/negative control absorbance]×100

TABLE 9

| | Pancreatic lipase inhibition rate (%) |
|---|---|
| CAB701 | 28.87 ± 4.8 |
| 1000 μg/mL orlistat | 28.55 ± 6.2 |

Looking at Table 9, it can be seen that the CAB701 strain has a pancreatic lipase inhibition rate of 28.87%, which is almost identical to the inhibition rate of 1000 μg/mL orlistat.

### 6-2: Cytotoxicity

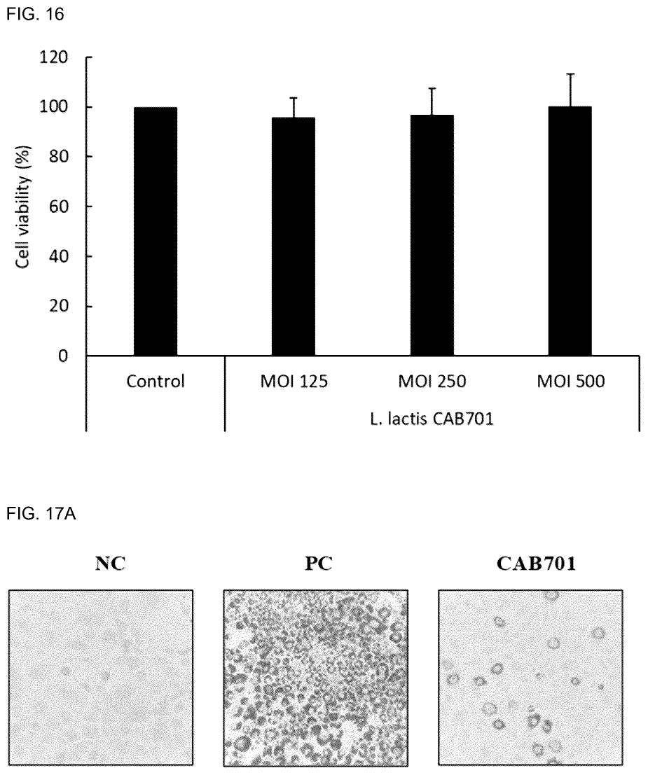

The viability of 3T3-L1 preadipocytes was measured in the same way as the RAW 264.7 cells. Specifically, 3Te-L1 cells were inoculated at a cell density of 1×10⁴ cells/well, and treated with the CAB701 strain at MOI 125, MOI 250 or MOI 500 concentrations. Then, cytotoxicity was measured using Ez-cytox (FIG. 16).

Referring to FIG. 16, the viability of the 3T3-L1 cells treated with the LAB701 strain was found to be MOI 125 (95.8%), MOI 250 (96.7%), and MOI 500 (100.31%), indicating that the LAB701 strain is non-cytotoxic.

### 6-3: Inhibition of Adipocyte Differentiation

3T3-L1 preadipocytes (ATCC CL-173) purchased from the American Type Culture Collection (ATCC; Rockville, MD, USA) were inoculated to DMEM containing 10% BCS (GIBCO) and 1% P/S, and cultured at 37° C. and 5% CO₂. For adipocyte differentiation, the 3T3-L1 cells were seeded at a density of 1×10⁵ cells/well in a 6-well plate and cultured for another 2 days after 100% confluency. After replacing the medium with a differentiation medium (MDI) consisting of DMEM supplemented with 10% fetal bovine serum (FBS; Corning Inc., Corning, NY, USA), 0.5 mM 3-Isobutyl-1-methylxanthine (IBMX, Sigma-Aldrich), 10 UM dexamethasone (Sigma-Aldrich), and 10 μg/mL insulin (Sigma-Aldrich), the cells were cultured for 3 days. The cells were then cultured for another 2 days after replacing the medium with an insulin medium consisting of DMEM containing 10% FBS and 10 μg/mL insulin. Then, the medium was replaced with a DMEM medium containing 10% FBS on days 5 and 7. Lactobacilli were treated at a concentration of MOI 500 when the medium was exchanged.

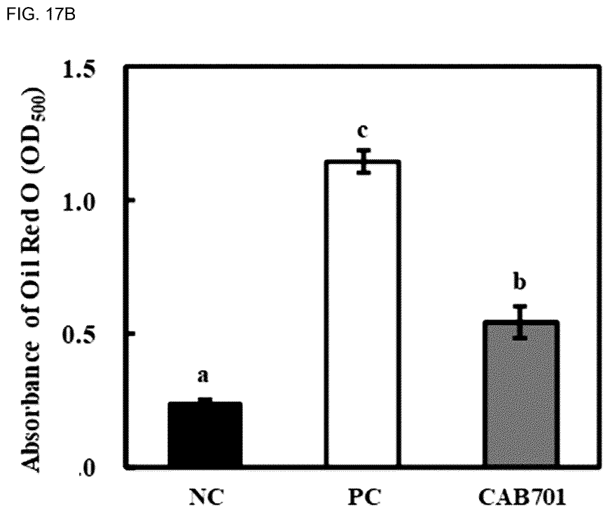

The differentiated 3T3-L1 cells were fixed with 10% formaldehyde solution for 20 minutes. After adding 0.3% oil red O solution (Sigma-Aldrich) to each well, the cells were stained for 15 minutes in dark (FIG. 17A). The cells were washed twice with PBS and, after adding 1 mL of 100% 2-propanol to each well, absorbance was measured at 500 nm to confirm differentiation into adipocytes (FIG. 17B). 3T3-L1 cells without MDI medium (differentiation medium) treatment were used as a negative control group, and 3T3-L1 cells treated with MDI medium (differentiation medium) without lactic acid bacteria treatment were used as a positive control group.

$$Adipocyte differentiation ( \% ) = [ ( sample absorbance - negative control absorbance ) / ( positive control group absorbance - negative control absorbance ) ] \times 100$$

Referring to FIGS. 17A and 17B, the group treated with the CAB701 strain showed an adipocyte differentiation rate of 38.53% as compared to the positive control group, indicating that the adipocyte differentiation rate was decreased by 60% or more.

### 6-4: Inhibition of Expression of Genes Involved in Fat Production

Since it was confirmed in Example 6-3 that the CAB701 strain has excellent ability of inhibiting adipocyte differentiation, the expression of adipogenesis-related genes was further analyzed.

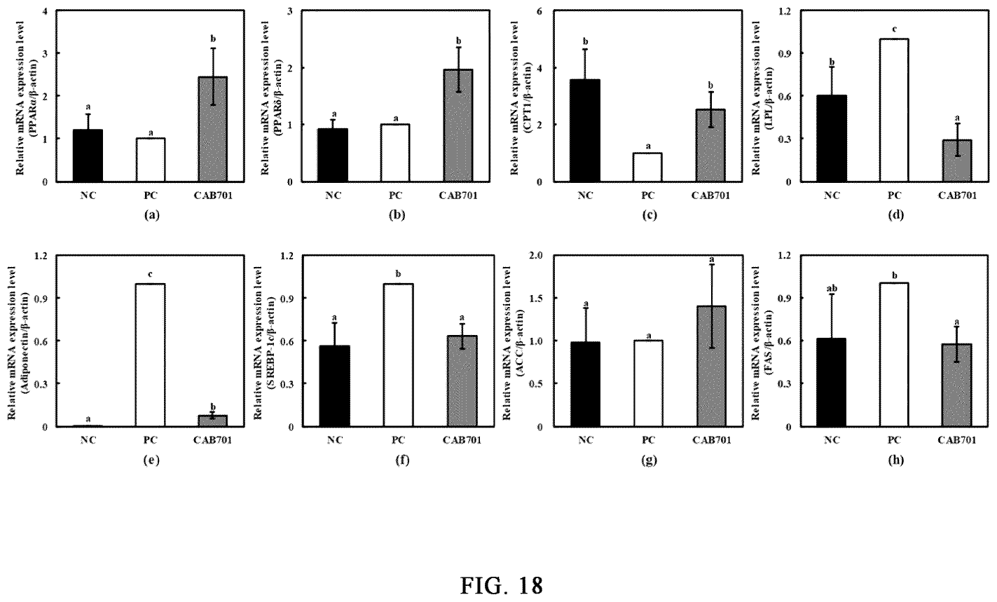

3T3-L1 cells were seeded in a 6-well plate at a density of 1×10⁵ cells/well, treated with lactic acid bacteria to an MOI of 500, and then cultured at 37° C. and 5% CO₂ for differentiation into mature adipocytes. RNA was extracted using the same method as for the RAW 264.7 cells and the mRNA expression level of adipogenesis-related genes in the 3T3-L1 cells was analyzed by RT-qPCR. The primer sequences and RT-qPCR conditions used are shown in Table 10 and Table 11, respectively. After normalization using the β-actin gene as a housekeeping gene, the relative mRNA expression of the adipogenesis-related genes was calculated from the ΔΔCT value. The result is shown in FIG. 18.

TABLE 10

| No. | Gene | | Primer sequence | Size (bp) |
|---|---|---|---|---|
| 7 | β-Actin | F | 5′-GAG CGC AAG TAC | 97 |
| | | | TCT GTG TG-3′ | |
| | | R | 5′-CGG ACT CAT CGT | |
| | | | ACT CCT G-3′ | |
| 8 | PPARα | F | 5′-GTA CGG TGT GTA | 76 |
| | | | TGA AGC CAT CTT-3′ | |
| | | R | 5′-GCC GTA CGC GAT | |
| | | | CAG CAT-3′ | |
| 9 | PPARδ | F | 5′-GCC ATA TTC CCA | 102 |
| | | | GGC TGT C-3′ | |
| | | R | 5′-CAG CAC AAG GGT | |
| | | | CAT CTG TG-3′ | |
| 10 | CPT1 | F | 5′-GTG ACT GGT GGG | 83 |
| | | | AGG AAT AC-3′ | |
| | | R | 5′-GAG CAT CTC CAT | |
| | | | GGC GTA G-3′ | |
| 11 | SREBP-1c | F | 5′-ACG GAG CCA TGG | 278 |
| | | | ATT GCA CA-3′ | |
| | | R | 5′-AAG GGT GCA GGT | |
| | | | GTC ACC TT-3′ | |
| 12 | ACC | F | 5′-ATG GGC GGA ATG | 148 |
| | | | GTC TCT TTC-3′ | |
| | | R | 5′-TGG GGA CCT TGT | |
| | | | CTT CAT CAT-3′ | |
| 13 | LPL | F | 5′-CCA CAG CAG CAA | 87 |
| | | | GAC CTT C-3′ | |
| | | R | 5′-AGG GCG GCC ACA | |
| | | | AGT TTG-3′ | |
| 14 | FAS | F | 5′-TGC TCC CAG CTG | 107 |
| | | | CAG GC-3′ | |
| | | R | 5′-GCC CGG TAG CTC | |
| | | | TGG GTG TA-3′ | |
| 15 | Adiponectin | F | 5′-GAG ATG CAG GTC | 105 |
| | | | TTC TTG GTC-3′ | |
| | | R | 5′-GCT CTC CTT TCC | |
| | | | TGC CAG-3′ | |

TABLE 11

| | | Temperature | | | Number |
|---|---|---|---|---|---|
| Step | | (° C.) | Time | | of cycles |
| Hold stage | | 50 | 2 | min | 1 |
| | | 95 | 2 | min | |
| PCR stage | Denaturation | 95 | 15 | sec | 35 |
| | Annealing | 56 | 30 | sec | |
| | Extension | 72 | 30 | sec | |

| Melting curve stage | 95 | 15 | sec | 1 |
|---|---|---|---|---|
| | 65 | 1 | min | |
| | 97 | 1 | sec | |

Lipoprotein lipase (LPL) is responsible for transporting fatty acids of lipoproteins into tissues, leading to the accumulation of fat in body tissues such as adipose tissue, etc. Other enzymes involved in fat synthesis include the fatty acid synthase (FAS) complex, etc. In addition, PPARa (peroxisome proliferator-activated receptor alpha) and CPT (carnitine palmitoyltransferase) are enzymes that promote fat oxidation. The increase in the oxidation rate of fatty acids may be a mechanism by which body fat is reduced.

Referring to FIG. 18, the 3T3-L1 cells treated with the CAB701 strain showed significant increase in the expression of the PPARa, PPARo and CPT1 genes, which are enzymes that promote fat oxidation, and significant decrease in the expression of the LPL, adiponectin and FAS genes, which are enzymes related to fat accumulation and fat synthesis, as compared to the positive control group.

### 6-5: Increased Expression of Fat Metabolism-Related Proteins

The effect of the CAB701 strain according to the present disclosure on the protein expression level of fat metabolism-related genes was analyzed by western blot.

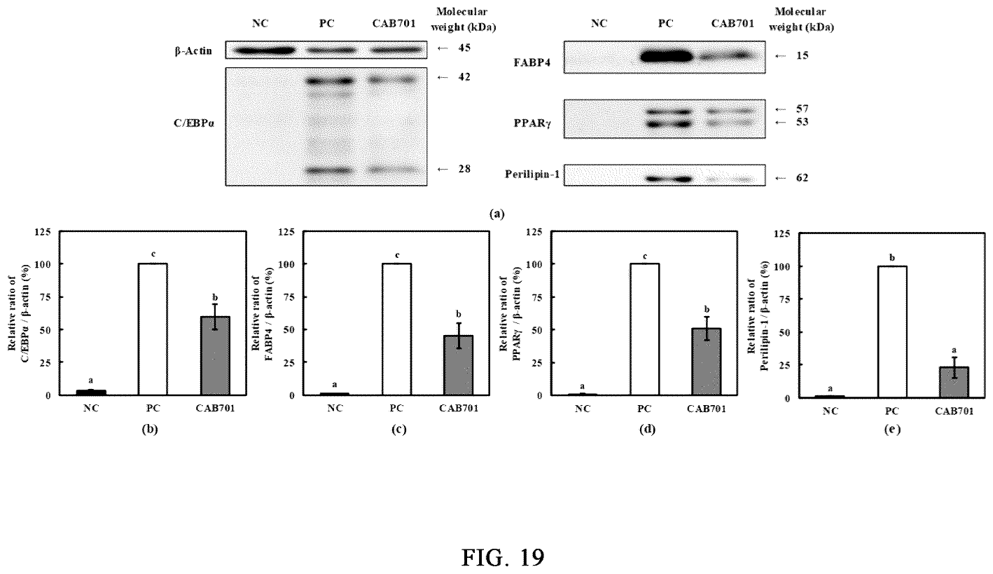

Specifically, 3T3-L1 cells were seeded in a 6-well plate at a concentration of 1×10⁵ cells/well, treated with lactic acid bacteria to an MOI of 500, and differentiated into mature adipocytes at 37° C. and 5% CO₂. After extracting proteins in the same way as for the RAW 264.7 cells, the expression level of fat metabolism-related proteins in the 3T3-L1 cells was analyzed by western blot (FIG. 19). For reference, because fatty acid binding proteins (FABPs) are responsible for transporting fatty acids and other lipids into various cellular pathways, decreased expression of FABPs can lead to decreased fat metabolism.

Referring to FIG. 19, it can be seen that the expression of C/EBPa, FABP4, PPARy and perilipin-1 was significantly decreased in the 3T3-L1 cells treated with the CAB701 strain as compared to the positive control group, indicating that the CAB701 strain modulates lipid metabolism in the 3T3-L1 cells, suggesting that it may exhibit anti-obesity activity.

### Example 7: Intestinal Stability of Strain

Probiotic strains are generally required to be acid-resistant, bile-resistant, pancreatic fluid-resistant, and intestinally adherent in order to survive in the stomach under strongly acidic condition and reach the small intestine in order to exert sufficient bioactivity. Therefore, the intestinal stability of the CAB701 strain, which was selected as a functional strain for immunity and anti-obesity, was analyzed.

### 7-1: Acid Resistance

Artificial gastric fluid was prepared by adjusting the pH of 100 mM glycine-HCl buffer to 2.5. After inoculating Lactobacillus seed culture incubated at 37° C. for 20 hours to 1% (v/v) in 4 mL of MRS broth, the culture was incubated at 37° C. for 16 hours. The culture was centrifuged at 13000 rpm for 1 minute and the obtained cell precipitate was washed twice with 0.88% (w/v) NaCl (sterile physiological saline). The washed pellet was resuspended in 4 mL of the artificial gastric fluid and incubated at 37° C. After 2 hours of incubation, 1 mL of the sample was taken and centrifuged at 13000 rpm for 1 minute to obtain a cell pellet, which was washed three times with sterile physiological saline and resuspended in 1 mL of sterile physiological saline. After decimally diluting with 9 mL saline, followed by smearing on LAB Petrifilm, the sample was incubated at 37° C. for 1-2 days and the live bacteria were counted (Table 12). Acid resistance was calculated using the following equation. The sample at 0 hour of incubation was used as a control group.

$$Acid resistance ( \% ) = [ log ( live bacterial count in sample ) / log ( live bacterial count in control group ) ] \times 100$$

TABLE 12

| | | Acid | | | Acid |
|---|---|---|---|---|---|
| CAB701 | | resistance | LGG (comparative strain) | | resistance |
| 0 h (CFU/mL) | 2 h (CFU/mL) | (%) | 0 h (CFU/mL) | 2 h (CFU/mL) | (%) |
| 4.60 × 10⁸ | 8.90 × 10³ | 45.6 | 9.30 × 10⁸ | 1.90 × 10⁷ | 81.2 |
| 4.20 × 10⁸ | 1.92 × 10⁴ | 49.6 | 8.30 × 10⁸ | 4.50 × 10⁷ | 85.8 |
| 3.10 × 10⁸ | 1.07 × 10⁴ | 47.5 | 1.01 × 10⁹ | 3.00 × 10⁷ | 83.0 |

| | | | |
|---|---|---|---|
| Average | 47.6 ± 2.0 | Average | 83.3 ± 2.3 |

As seen from Table 12, the acid resistance of the CAB701 strain of the present disclosure was found to be 47.6%, while the acid resistance of the comparative strain Lactobacillus rhamnosus GG, which is known to have good acid resistance, was found to be 83.3%.

### 7-2: Bile Resistance

Artificial bile was prepared by adding 3% of a 10% Bacto oxgall (Becton, Dickinson and Company) solution filtered through a 0.22-μm membrane to sterilized MRS broth to give a final oxgall concentration of 0.3%. A CAB701 strain seed culture obtained by incubating at 37° C. for 20 hours after inoculation to MRS broth was added to 5 mL of the artificial bile to 1% (v/v) and incubated at 37° C. for 24 hours. 1 mL of the sample was taken, diluted decimally with 9 mL of saline, smeared on LAB Petrifilm, and incubated at 37° C. for 1-2 days to determine the number of live bacteria (Table 13). Bile resistance was calculated using the following equation. A sample incubated in MRS broth without addition of oxgall was used as a control group.

$$Bile resistance ( \% ) = [ log ( live bacterial count in sample ) / log ( live bacterial count in control group ) ] \times 100$$

TABLE 13

| | Bile resistance (%) |
|---|---|
| CAB701 | 88.2 ± 0.8 |
| LGG (comparative strain) | 63.6 ± 9.3 |

From Table 13, it can be seen that the bile resistance of the CAB701 strain of the present disclosure is 63.6%, indicating good bile resistance.

### 7-3: Pancreatic Juice Resistance

Artificial pancreatic juice was prepared by adding 5% of a 10% pancreatin solution (0.85% NaCl, pH 8.0) filtered through a 0.22-μm membrane to sterilized MRS broth to a final pancreatin concentration of 0.5%. A Lactobacillus seed culture obtained by incubating at 37° C. for 20 hours after inoculation to MRS broth was added to 5 mL of the artificial pancreatic juice to 1% (v/v) and incubated at 37° C. After 3 hours of incubation, 1 mL of the sample was taken, diluted decimally with 9 mL of saline, smeared on LAB Petrifilm, and incubated at 37° C. for 1-2 days to determine the number of live bacteria (Table 14). Pancreatic juice resistance was calculated using the following equation. A sample at 0 hour of incubation was used as a control group.

$$Pancreatic juice resistance ( \% ) = [ log ( live bacterial count in sample ) / log ( live bacterial count in control group ) ] \times 100$$

TABLE 14

| | | | LGG (comparative | | |
|---|---|---|---|---|---|
| CAB701 | | | strain) | | |
| 0 h | 2 h | Pancreatic juice | 0 h | 2 h | Pancreatic juice |
| (CFU/mL) | (CFU/mL) | resistance (%) | (CFU/mL) | (CFU/mL) | resistance (%) |
| 1.38 × 10⁹ | 2.00 × 10⁹ | 101.8 | 1.30 × 10⁹ | 3.14 × 10⁹ | 104.2 |
| 1.21 × 10⁹ | 1.94 × 10⁹ | 102.3 | 1.73 × 10⁹ | 3.87 × 10⁹ | 103.8 |
| 1.37 × 10⁹ | 1.98 × 10⁹ | 101.8 | 1.12 × 10⁹ | 2.74 × 10⁹ | 104.3 |

| | | | |
|---|---|---|---|
| Average | 101.9 ± 0.3 | Average | 104.1 ± 0.3 |

As seen from Table 14, the pancreatic juice resistance of the CAB701 strain of the present disclosure was 101.9%, and the pancreatic juice resistance of the LGG strain was 104.1%. Both strains exhibited high pancreatic juice resistance exceeding 100%.

### 7-4: Ability of Intestinal Adherence

Caco-2 cells (human colorectal adenocarcinoma cells, HTB-37™), which are enterocyte-like cells, purchased from the ATCC were cultured in DMEM medium supplemented with 10% (v/v) FBS (Cornig) and 1% (v/v) P/S at 37° C. under 5% CO₂ in an incubator (Thermo Fisher Scientific, Waltham, MA, USA). 0.25% trypsin-EDTA (GIBCO) was used to detach the cells from a culture flask.

500 μL of the Caco-2 cells were seeded in a 24-well plate at a density of 5×10⁵ cells/well and cultured for 3 days at 37° C. and 5% CO₂. The 24-well plate in which the Caco-2 cells were cultured was washed twice with 500 μL of DMEM medium supplemented with 10% FBS. After treating with Lactobacilli at a concentration of MOI 200, the cells were incubated at 37° C. and 5% CO₂ for 2 hours. After the incubation, each well was washed four times with 500 μL of DPBS to remove the unattached strain. 200 μL of 0.25% trypsin-EDTA was dispensed into each well and incubated for 5 minutes at 37° C. and 5% CO₂. The detached Caco-2 cells and strain were collected in a 1.5-mL tube together with 300 L of DPBS and centrifuged at 13,000 rpm for 1 minute to harvest a cell pellet, which was washed twice with 1 mL of DPBS. The cell pellet was resuspended in 1 mL of 0.1% (v/v) Triton X-100 diluted in DPBS and dispensed into 9 mL of saline. After decimal dilution, the cell pellet was smeared on LAB Petrifilm and incubated at 37° C. for 1-2 days to determine the number of live bacteria (Table 15). As a control group, the strain was suspended in DMEM medium supplemented with 10% FBS and incubated at 37° C. for 2 hours. Then, 500 μL of the culture was dispensed into 9.5 mL of saline. After decimal dilution, followed by smearing on Petrifilm and incubation at 37° C. for 24 hours, the number of live bacteria was determined. The ability of intestinal adhesion was calculated by the following equation.

$$Ability of intestinal adherence ( \% ) = [ log ( live bacterial count in sample ) / log ( live bacterial count in control group ) ] \times 100$$

TABLE 15

| | Ability of intestinal adherence (%) |
|---|---|
| CAB701 | 77.2 ± 0.8 |
| LGG (comparative strain) | 75.5 ± 9.3 |

As seen from Table 15, there was no significant difference in the ability of intestinal adherence of the LGG (75.5%) and CAB701 (77.2%) strains.

### Example 8: Evaluation of Safety of Strain

### 8-1: Antibiotic Resistance

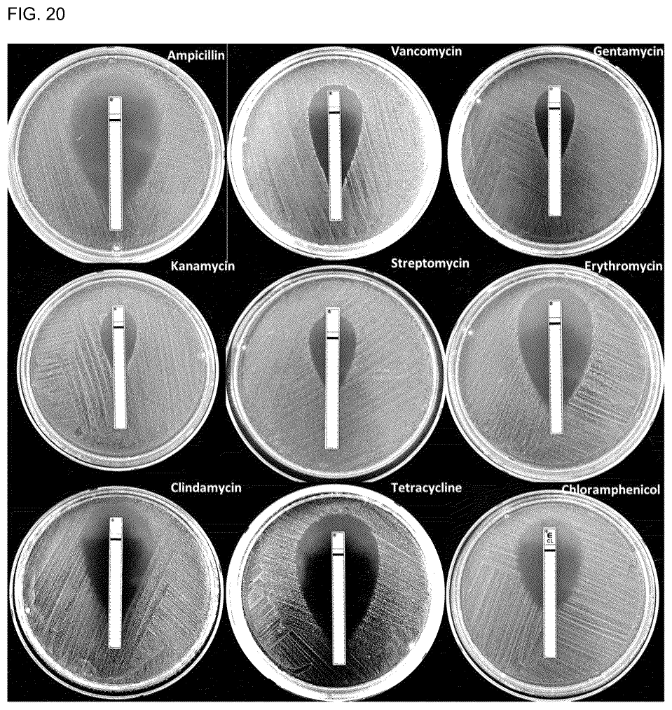

The antibiotic resistance of the L. lactis CAB701 strain was evaluated by E-test (bioMeurieux, La Balme-Les-Grottes, France). 100 μL of a CAB701 strain culture was smeared on an antibiotic-free MRS agar plate adjusted to the turbidity of 3 McFarland standard (0.03% barium chloride, 5.97% sulfuric acid), located at the center of an E-test strip on a plate, and incubated at 37° C. for 72 hours. The value at the end of the strip pointed by the inhibitory ring was determined as the minimum inhibitory concentration (MIC). The cutoff values for ampicillin, vancomycin, gentamycin, kanamycin, streptomycin, erythromycin, clindamycin, tetracycline and chloramphenicol were set in accordance with the reference values proposed by the EFSA (FIG. 20 and Table 16).

Referring to FIG. 20, the minimum inhibitory concentration (MIC) of the CAB701 strain for ampicillin, vancomycin, gentamycin, kanamycin, streptomycin, erythromycin, clindamycin, tetracycline and chloramphenicol were 0.094 μg/mL, 0.38 μg/mL, 3 g/mL, 8 μg/mL, 16 μg/mL, 0.125 μg/mL, 0.25 μg/mL, 0.094 μg/mL and 1.5 μg/mL, respectively, which were all below the cutoff values, confirming that the strain is not resistant to the antibiotics.

TABLE 16

| Antibiotics | Cut-off value (μg/mL) | MIC (μg/mL) | Evaluation |
|---|---|---|---|
| Ampicillin | 2 | 0.094 | Allowed |
| Vancomycin | 4 | 0.38 | Allowed |
| Gentamicin | 32 | 3 | Allowed |
| Kanamycin | 64 | 8 | Allowed |
| Streptomycin | 2 | 16 | Allowed |
| Erythromycin | 1 | 0.125 | Allowed |
| Clindamycin | 1 | 0.25 | Allowed |
| Tetracycline | 4 | 0.094 | Allowed |
| Chloramphenicol | 8 | 1.5 | Allowed |

### 8-2: Effect on D-Lactate Production

There are two types optical isomers of lactic acid, which are L-form and D-form. The human body can metabolize the L-form, but not the D-form. Some probiotic strains have D/L-lactate racemase, an enzyme that converts L-lactic acid to D-lactic acid. If the activity of this enzyme is strong, D-lactate can accumulate and cause acidosis in newborns, children, or patients with short bowel syndrome. Therefore, the WHO recommends a daily intake of D-lactic acid of no more than 100 mg/kg body mass.

The CAB701 strain of the present disclosure was cultured in MRS broth at 37° C. for 24 hours, and the concentration of D-lactic acid and L-lactic acid in the culture was measured using a D-lactic acid assay kit (Roche).

TABLE 17

| | D-Lactic acid | L-Lactic acid | D-Lactic acid/ |
|---|---|---|---|
| | (mM) | (mM) | L-lactic acid |
| CAB701 | — | 5.41 | — |

Looking at Table 17, it can be seen that the CAB701 strain of the present disclosure produces L-lactic acid only, and there is no safety issue related to the production of D-lactic acid.

### 8-3: Enzyme Activity-API ZYM Test

Enzyme activity was analyzed for the CAB701 strain using an API ZYM kit (bioMerieux, Marcy l′Etoile, France).

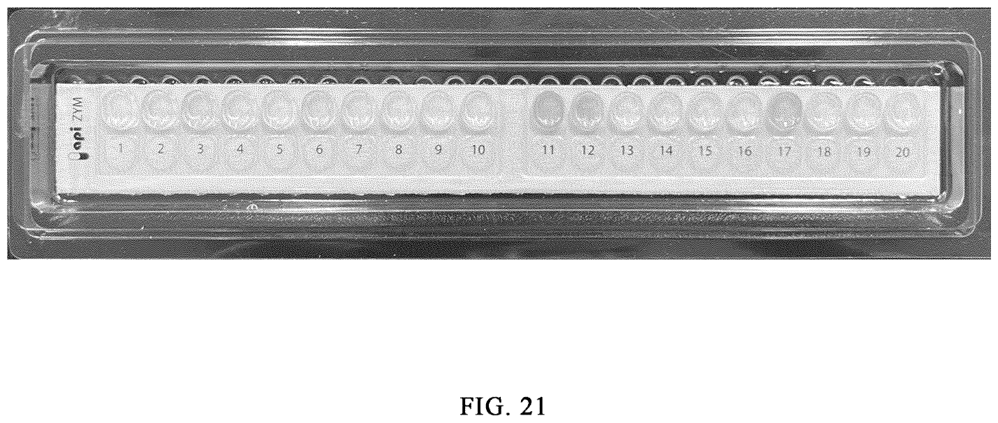

Specifically, the CAB701 strain of the present disclosure was incubated for 24 hours and then diluted in 2 mL of sterile physiological saline to adjust turbidity to Mcfarland 5-6. After pouring 5 mL of sterile physiological saline into a culture box and dispensing 65 μL of the turbidity-adjusted strain on an API strip, the strain was incubated at 37° C. for 4 hours. Then, after adding ZYM A and ZYM B drop by drop, color change was checked 5 minutes later (FIG. 21 and Table 18).

Referring to FIG. 21 and Table 18, when ZYM A and ZYM B were dropped onto the API ZYM kit in which the CAB701 strain was cultured, enzymatic activity was observed against acid posphatase, naphtol-AS-BI-phosphohydrolase and β-glucosidase. But, no activity was observed against β-glucuronidase, which is an enzyme that converts benzopyrene into a carcinogenic substance, confirming that the strain is safe.

TABLE 18

| | Enzyme | Reaction |
|---|---|---|
| 1 | Control | − |
| 2 | Alkaline phosphatase | − |
| 3 | Esterase (C4) | − |
| 4 | Esterase lipase (C8) | − |
| 5 | Lipase (C14) | − |
| 6 | Leucine arylamdiase | − |
| 7 | Valine arylamdiase | − |
| 8 | Crystine arylamdiase | − |
| 9 | Trypsin | − |
| 10 | α-Chymotrypsin | − |
| 11 | Acid phospatase | + |
| 12 | Naphtol-AS-BI-phosphohydrolase | + |
| 13 | α-Galactosidase | − |
| 14 | β-Galactosidase | − |
| 15 | β-Glucuronidase | − |
| 16 | α-Glucosidase | − |
| 17 | β-Glucosidase | + |
| 18 | N-Acetyl-β-glucosaminidase | − |
| 19 | α-Mannosidase | − |
| 20 | α-Fucosidase | − |

### Sub-Conclusion

Among the 58 strains selected from the eight types of Jeju raw materials (carrot, cactus, broccoli, beet, cabbage, hallabong, kale and kohlrabi), the cabbage-derived Lactococcus lactis subsp. lactis CAB701 strain, which is a strain notified by the Ministry of Food and Drug Safety as a health functional probiotic, was finally selected for its anti-obesity and immune-enhancing activities.

The NO production-promoting activity of the selected CAB701 strain was found to be very high. In addition, as a result of investigating the expression level of cytokine-related genes (iNOS, COX-2, TNF-α, IL-1β and IL-6) and proteins related to the MAPKs signaling pathway the CAB701 strain in RAW 264.7 cells, it was found to have superior immune-enhancing activity.

In addition, it was specifically confirmed that the CAB701 strain has excellent pancreatic lipase inhibitory activity, inhibits adipocyte differentiation of 3T3-L1 cells, suppresses the expression of adipogenesis-related genes, and increases the expression of fat metabolism-related proteins.

Taken together, it was confirmed that the CAB701 strain isolated and identified in the examples of the present disclosure has high NO production-promoting activity, increases the expression of cytokine-related genes (iNOS, COX-2, TNF-α, IL-1β and IL-6) and the expression of MAPKs signaling pathway-related proteins, exhibits good pancreatic lipase inhibitory activity, inhibits adipocyte differentiation of 3T3-L1 cells, suppresses the expression of adipogenesis-related genes, and increases the expression of fat metabolism-related proteins.

Accordingly, the inventors of the present disclosure have deposited the strain at the Korean Culture Center of Microorganisms (KCCM) on Jun. 16, 2023, and have been assigned the following accession number.

Lactococcus lactis subsp. lactis-Accession No. KCCM 13360P (also named “CAB701”)

### <Evaluation of Function In Vivo>

### Example 9: Anti-Obesity Activity

### Experimental Methods

### Experimental Animals

As experimental animals, 5-week-old C57BL/6 mice (male) were obtained from Hana Bio (Gyeonggi-do, Korea) and maintained in the animal house of the SouthEast Medi-chem Institute (Animal Facility Registration No. 412) after quarantine and a week of acclimatization and breeding. During rearing, lighting was set to 12 hours (from 07:00 to 19:00), and feed and drinking water were allowed ad libitum. The experimental animals were grouped and treated according to the design shown in Table 19 below. The study was conducted in accordance with the policies and regulations of the Institutional Animal Care and Use Committee (SEMI-23-003) of the SouthEast Medi-chem Institute.

TABLE 19

| | | | Route of | |
|---|---|---|---|---|
| | | Adminis- | adminis- | Induction of |
| | n | tration | tration | obesity |
| Normal | 10 | Saline | Oral administration | −(normal diet) |
| group (Normal) | | | (dosage: | |
| | | | 10 mL/kg) | |
| Obesity control | 10 | Saline | | +(60 HFD) |
| group (control) | | | | |
| Test group | 10 | CAB701 500 | | +(60 HFD) |
| (sample) | | mg/kg | | |

### Administration of Sample

The CAB701 strain was dissolved in saline (0.9% NaCl) to a concentration of 500 mg/kg and administered orally for 6 weeks.

Induction of Obesity and Treatment with Test Substance

A normal group (hereinafter, referred to as Normal) was allowed free access to a basal AlN 93G diet. To induce obesity, all groups except the normal group were fed a 60% high-fat diet (hereinafter, referred to as HFD, 60% fat per kcal, Research Diets Inc., New Brunswick, NJ, USA) ad libitum for 6 weeks and orally administered with the CAB701 strain sample of the present disclosure at the same time. Body weight, feed intake and drinking water consumption were measured 3 times per week during the administration of the CAB701 strain sample.

### End of Experiment and Biological Sampling

After the administration of the CAB701 strain sample, the obesity-induced experimental animals were treated with CO₂ and blood was collected from the abdominal vena cava. The blood was collected in a serum separation tube (SST) and allowed to stand at room temperature for approximately 30 minutes before centrifugation at 3,500 rpm for 15 minutes to separate the serum. To compare fat mass between the groups, periepididymal fat was harvested and weighed.

### Analysis of Biochemical Indices of Serum

The serum separated after the blood collection was tested for TG (triglycerides), TC (total cholesterol), HDL-C(high denesitiy lipoprotein cholesterol), and LDL-C(low denesitiy lipoprotein cholesterol), which are lipid marker enzymes, using an automated biochemical analyzer (7100, Hitachi, Japan).

### Analysis of Triglycerides in Feces

Triglyceride content in feces was determined using a modified method of Folch et al. (1957). Dried feces were ground in a mortar. 0.2 g was taken and extracted at 4° C. for 24 hours by adding 5 mL of chloroform:methanol (2:1. v/v) solution. The extract was centrifuged at 3000 rpm and 4° C. for 10 minutes, and the supernatant was taken, dried and then re-dissolved in 0.5 mL of the same extraction solvent. After drying and dissolving in ethanol, 0.2 mL was taken for measurement of triglycerides in order to determine the triglyceride content in the feces.

### Analysis of Protein Expression Using Periepididymal Fat: Western Blot

After adding RIPA buffer to periepididymal fat, it was homogenized using a mini-homogenizer. After centrifugation at 4° C. and 12,000 rpm for 20 minutes, the supernatant was separated. The separated supernatant was subjected to protein quantification by the Bradford method followed by separation based on size by SDS-PAGE (polyacrylamide gel electrophoresis). Proteins were transferred onto a nitrocellulose membrane using a semi-dry transfer system (Biorad, USA) and treated for 1 hour in a blocking buffer containing 5% skim milk. As primary antibodies, FAS (Cat. No. sc-74540, Santacruz, USA), PPAR-y (Cat. No. sc-7273, Santacruz, USA) and β-actin (Cat. No. sc-47778, Santacruz, USA) were treated at a ratio of 1:1000 and reaction was conducted at 4° C. After reacting with the membrane using a western blot detection kit, the expression of the bands was observed using Chemi-Doc equipment (Biorad, USA) and the result was compared by calibrating with β-actin.

### Histopathological Analysis

Periepididymal adipose tissue was fixed and prepared into paraffin blocks for sectioning. After deparaffinization, the sections were dehydrated and stained with H&E (hematoxylin & eosin). After being embedding into a mounting medium, the sections were imaged at magnifications of 40× and 100× using an ordinary optical microscope (E600, Nikon, Japan), and adipocyte size was measured using the Image J program (Java-based image processing program, USA).

### Statistical Analysis

Statistical test was performed using the Statview (ver. 5.0.1) statistical program. The measurement results were expressed as mean±standard deviation. Statistical significance was post-tested by Fisher's PLSD at the p<0.05 after the ANOVA test.

### Test Results

### 9-1: Body Weight Change and Induction of Obesity

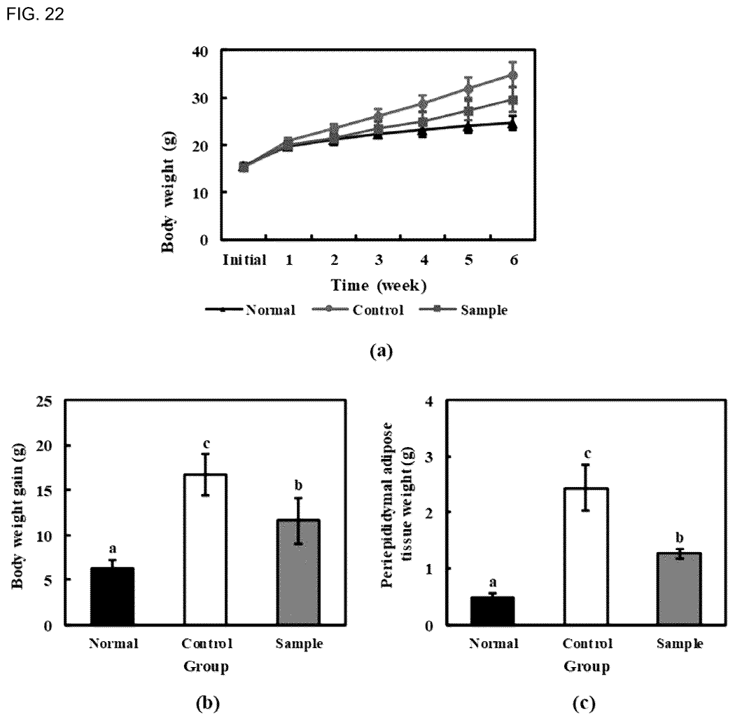

Except for the normal group, all other groups were administered with the test substance (CAB701 strain) together with the HFD diet for 6 weeks. As a result of investigating body weight change, body weight gains was observed for the obesity control group (Control) fed with the HFD as compared to the normal group (Normal), and the test group (Sample) showed significant decrease in body weight as compared to the obesity control group (Control) (FIG. 22 and Table 20).

TABLE 20

| | Initial body | Final body |
|---|---|---|
| | weight (g) | weight (g) |
| Normal group (Normal) | 15.572 ± 0.302 | 24.631 ± 1.440 |
| Obesity control | 15.499 ± 0.640 | 34.912 ± 2.542* |
| group (Control) | | |
| Test group (Sample) | 15.486 ± 0.592 | 29.623 ± 2.501^{#} |

### 9-2: Adipose Tissue Weight

The periepididymal adipose tissue weight was increased in all the groups fed the high-fat diet increased as compared to the normal group. The test group to which the test substance (CAB701 strain) was treated showed significant decrease in the periepididymal adipose tissue weight with p<0.05 as compared to the obesity control group (Control) (FIG. 22 and Table 21).

TABLE 21

| | | Periepididymal adipose |
|---|---|---|
| | n | tissue weight (g) |
| Normal group (Normal) | 10 | 0.475 ± 0.090 |
| Obesity control | 10 | 2.436 ± 0.406* |
| group (Control) | | |
| Test group (Sample) | 10 | 1.267 ± 0.081^{#} |

### 9-3: Analysis of Biochemical Indices of Serum

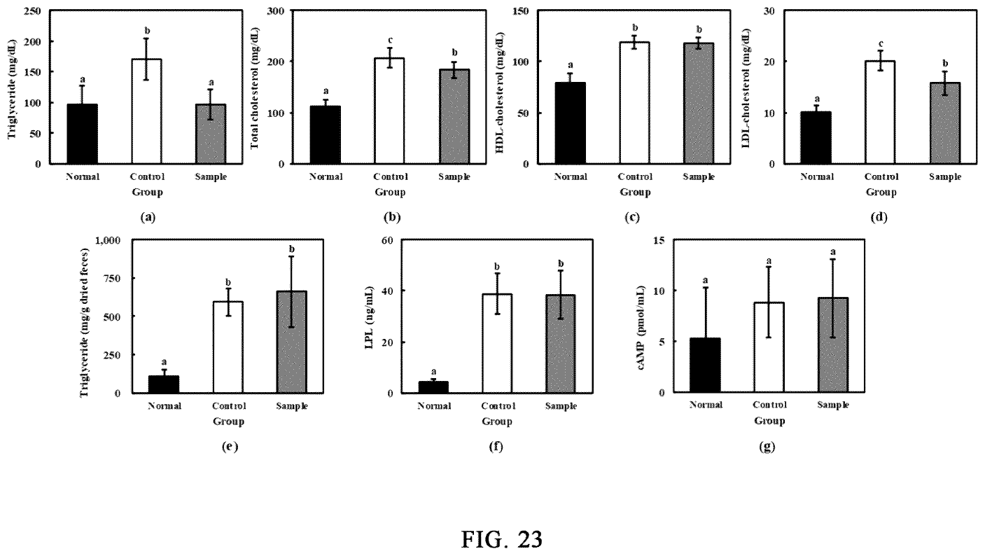

The biochemistry analysis of serum confirmed that all biochemical indices were increased significantly (p<0.05) in the HFD-induced obesity control group (Control) as compared to the normal group (Normal). On the other hand, the group (Sample) treated with the test substance (CAB701 strain) showed significant decrease in blood triglyceride, total cholesterol and LDL-cholesterol concentrations, which are indices of lipid metabolism, at p<0.05, as compared to the obesity control group (Control) (FIG. 23 and Table 22).

TABLE 22

| | | | Total | HDL | LDL |
|---|---|---|---|---|---|
| | | Triglycerides | cholesterol | cholesterol | cholesterol |
| | n | (mg/dL) | (mg/dL) | (mg/dL) | (mg/dL) |
| Normal | 10 | 116.73 ± 19.33 | 104.65 ± 11.98 | 85.62 ± 6.03 | 9.69 ± 1.21 |
| group (Normal) | | | | | |
| Obesity control | 10 | 157.99 ± 22.85* | 219.17 ± 14.40* | 115.31 ± 3.35* | 20.40 ± 1.96* |
| group (Control) | | | | | |
| Test group | 10 | 89.67 ± 22.16^{#} | 179.58 ± 14.95^{#} | 120.35 ± 2.95 | 14.88 ± 1.36^{#} |
| (Sample) | | | | | |

### 9-4: Analysis of Expression of Proteins of Lipogenic Factors in Periepididymal Adipose Tissue

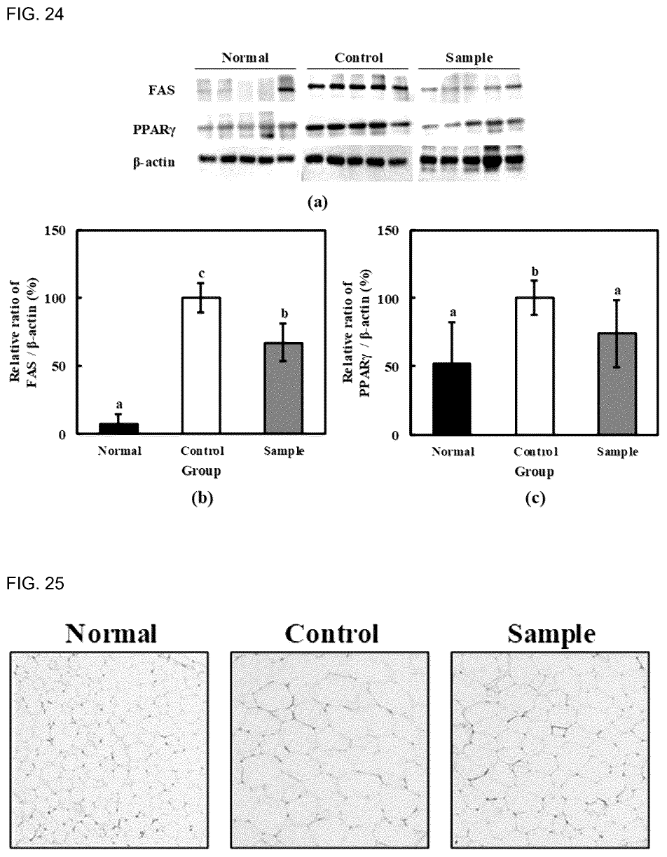

The HFD-induced obesity control group (Control) showed significant increase in the expression of FAS and PPAR-y as compared to the normal group (Normal) after 6 weeks of HFD administration, while the expression of FAS and PPAR-y was significantly (p<0.05) decreased in the group (Sample) treated with the test substance (CAB701 strain) as compared to the obesity control group (Control) (FIG. 24 and Table 23).

TABLE 23

| | n | FAS/β-actin | PPAR-γ/β-actin |
|---|---|---|---|
| Normal group (Normal) | 5 | 7.9 ± 6.7 | 52.3 ± 16.4 |
| Obesity control group (Control) | 5 | 100 ± 11.1* | 100 ± 7.6* |
| Test group (Sample) | 5 | 67.0 ± 14.1^{#} | 73.9 ± 10.0^{#} |

### 9-5: Investigation of Change in Adipocyte Size by Histologic Observation

Mouse periepididymal adipose tissue was stained with H&E and observed under an optical microscope to determine adipocyte size.

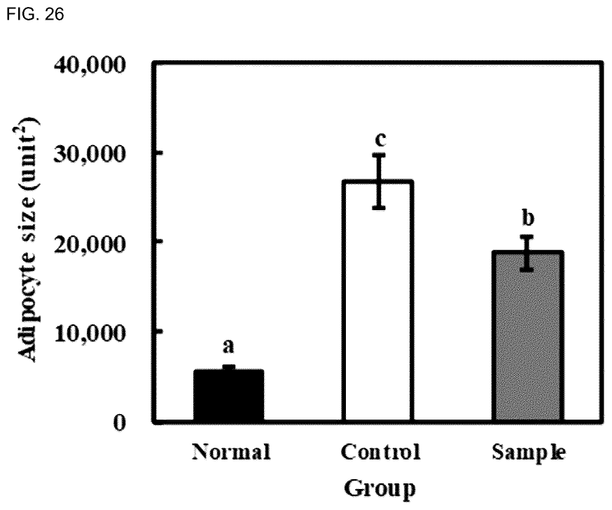

The HFD-induced obesity control group (Control) showed significant increase in adipocyte size as compared to the normal group (Normal) after 6 weeks of HFD administration, while the group (Sample) treated with the test substance (CAB701 strain) showed significant decrease in adipocyte size as compared to the obesity control group (Control) (FIG. 25, FIG. 26 and Table 24).

TABLE 24

| | n | Adipocyte size (units²) |
|---|---|---|
| Normal group (Normal) | 10 | 5,548.4 ± 561.9 |
| Obesity control group (Control) | 10 | 26,809.3 ± 2909.3* |
| Test group (Sample) | 10 | 18,816.5 ± 1867.5^{#} |

### 9-7: Sub-Conclusion

It was found that the administration of the CAB701 strain of the present disclosure inhibits body fat synthesis. Specifically, the test group to which the CAB701 strain of the present disclosure was administered showed significantly reduced body weight and epididymal fat weight, significantly reduced blood triglyceride, total cholesterol, and LDL-cholesterol concentrations, significantly reduced FAS and PPAR-y protein expression, which are known as lipogenic factors, and significantly reduced adipocyte size in epididymal adipose tissue, as compared to the obesity control group.

### <Clinical Test>

### Example 10: Anti-Obesity Effect

### Experimental Methods

### Selection of Subjects

100 healthy individuals (50 for a test group and 50 for a control group) with ages between 19 and 75 with a body mass index (BMI) between 23 and 30 kg/m² were selected.

### Preparation of Hard Capsule

For the test group, 10×10¹⁰ CFU/g of the CAB701 strain of the present disclosure, 3 mg of crystalline cellulose, and 14.8 mg of lactose were mixed and filled into a gelatin capsule, and a capsule (400 mg) was prepared according to a conventional hard capsule preparation method. And, for the control group, a capsule was prepared in the same manner, except that maltodextrin powder was used instead of the CAB701 strain.

### Test Methods

The 100 test subjects were asked to eat the same dietary meal as much as possible every day throughout the test period. The 50 subjects in the test group were given the capsule containing the CAB701 strain, once daily, with water after meals for 12 weeks (84 days). And, the 50 subjects in the control group were given the capsule containing the maltodextrin powder in the same manner.

### Statistical Analysis

Statistical test was performed using the Statview (ver. 5.0.1) statistical program. The measurement results were expressed as mean±standard deviation. Statistical significance was post-tested by Fisher's PLSD at the p<0.05 after the ANOVA test.

### Test Results

### 10-1: Anti-Obesity Effect

The DEXA body fat mass, body mass index (BMI), weight, and waist circumference of the test subjects were measured at the end of the 12 weeks of administration and the result is shown in Table 25. The DEXA body fat mass was measured by dual-energy X-ray absorptiometry (DEXA).

TABLE 25

| | | Control group | Test group |
|---|---|---|---|
| DEXA | Week 0 | 26.76 ± 3.65 | 26.38 ± 3.78 |
| Body fat (kg) | Week 12 | 27.46 ± 3.93* | 24.57 ± 3.68^{##} |
| | Amount of change | 0.70 ± 0.91 (2.61%) | −1.81 ± 2.28 (−6.86%) |
| DEXA | Week 0 | 38.99 ± 5.42 | 40.81 ± 4.90 |
| Body fat percentage | Week 12 | 39.76 ± 5.53* | 38.47 ± 4.90^{##} |
| (%) | Amount of change | 0.76 ± 1.13 (1.97%) | −2.34 ± 1.68 (−5.73%) |
| Body mass index | Week 0 | 25.39 ± 2.01 | 25.13 ± 1.66 |
| (BMI, kg/m²) | Week 12 | 25.80 ± 2.04* | 24.76 ± 1.93^{#} |
| | Amount of change | 0.41 ± 0.45 (1.61%) | −0.37 ± 0.80 (−1.48%) |
| Body weight (kg) | Week 0 | 69.30 ± 9.23 | 64.33 ± 6.63 |
| | Week 12 | 70.91 ± 9.60* | 63.06 ± 7.10^{#} |
| | Amount of change | 1.61 ± 1.31 (2.65%) | −1.27 ± 2.13 (−1.97%) |
| Waist circumference | Week 0 | 84.88 ± 7.39 | 81.55 ± 5.49 |
| (cm) | Week 12 | 84.21 ± 7.52 | 79.35 ± 5.64^{#} |
| | Amount of change | −0.67 ± 3.43 | −2.20 ± 3.45 (−2.7%) |
| Blood triglycerides | Week 0 | 102.63 ± 39.56 | 116.72 ± 91.32 |
| (mg/dL) | Week 12 | 114.78 ± 52.13* | 109.51 ± 54.52^{#} |
| | Amount of change | 12.15 ± 48.32 (11.84%) | −7.21 ± 68.76 (−6.18%) |
| Blood HDL (mg/dL) | Week 0 | 60.04 ± 10.78 | 62.19 ± 13.36 |
| | Week 12 | 59.46 ± 12.42 | 62.94 ± 13.34 |
| | Amount of change | −0.59 ± 9.03 | 0.74 ± 9.06 |
| Blood LDL (mg/dL) | Week 0 | 112.30 ± 36.14 | 116.21 ± 29.13 |
| | Week 12 | 107.24 ± 28.65 | 112.72 ± 29.88 |
| | Amount of change | −5.07 ± 32.43 | −3.49 ± 17.83 |
| Blood total | Week 0 | 194.41 ± 41.60 | 205.26 ± 37.02 |
| cholesterol (mg/dL) | Week 12 | 190.89 ± 33.32 | 199.11 ± 33.24 |
| | Amount of change | −3.52 ± 35.10 | −6.15 ± 26.06 |
| FFA (μmol/L) | Week 0 | 405.80 ± 206.60 | 468.91 ± 238.83 |
| | Week 12 | 470.48 ± 201.04* | 518.74 ± 230.39^{#} |
| | Amount of change | 64.67 ± 153.14 (15.94%) | 49.83 ± 273.47 (10.63%) |
| Adiponectin (pg/mL) | Week 0 | 5,784 ± 3,324 | 5,376 ± 3,253 |
| | Week 12 | 4,942 ± 2,758* | 5,955 ± 3,658^{#} |
| | Amount of change | −842 ± 2,404 (−14.56%) | 479 ± 2,432 (10.77%) |

Referring to Table 25, it can be seen that the test group to which the capsule containing the CAB701 strain according to the present disclosure was administered showed significant reduction in body fat mass and body fat percentage. Furthermore, the test group to which the capsule containing the CAB701 strain according to the present disclosure was administered showed significant reduction in the body mass index, body weight, waist circumference, and blood triglyceride content, and significant increase in the blood adiponectin content.

### 10-2: Sub-Conclusion

It was confirmed that the administration of the CAB701 strain of the present disclosure has the effect of reducing body fat. Specifically, the test group to which the capsule containing the CAB701 strain according to the present disclosure was administered showed significantly reduced body mass index, body weight, waist circumference, and blood triglycerides, significantly reduced blood triglyceride, total cholesterol, and LDL-cholesterol concentrations, and significantly increased blood adiponectin content, as compared to the control group.

### Example 11: Preparation and Fermentation Characteristics of Coconut Fermented Milk Using Strain

### 11-1: Preparation of Coconut Fermented Milk

(1) The CAB701 strain was inoculated to MRS broth and incubated at 37° C. Then, cells were harvested by centrifuging the Lactobacillus culture (13,000 rpm, 1 minute, 4° C.). The cell precipitate was washed 3 times with 0.85% (w/v) NaCl (sterile physiological saline) and then suspended in 1 mL of sterile physiological saline (The concentration of the suspension was 1×10⁸ CFU/mL).

(2) 1% (v/v) of the suspended strain was inoculated to 3 mL of coconut milk with a solid content of 8 wt %, and coconut fermented milk was prepared by fermenting at 37° C. for 16 hours.

### 11-2: Measurement of Number of Live Bacteria and pH of Coconut Fermented Milk

After measuring the pH of the coconut fermented milk prepared in Example 11-1, it was decimally diluted with sterile physiological saline and the number of live bacteria was counted on a Petrifilm™ lactic acid bacteria count plate (3M Company, St. Paul, MN, USA) after culturing for 1 to 2 days.

TABLE 26

| | LGG | CAB701 |
|---|---|---|
| Number of live bacteria (log CFU/mL) | 8.85 ± 0.18 | 9.09 ± 0.02 |
| pH | 3.57 ± 0.06 | 4.15 ± 0.02 |

Looking at Table 26, the coconut milk fermented with LGG had a low pH of 3.57, while that fermented with the CAB701 strain exhibited a pH between 4.1 and 4.2. And, the number of live bacteria of the coconut milk was approximately 109 CFU/mL for both the LGG and CAB701 strains.

### 11-3: Quality Characteristics of Coconut Fermented Milk

### 11-3-1: Lactobacillus Count Depending on pH

The adequate acidity of vegan lactic acid-fermented milk is around 2.5.

The fermentation was ended when the coconut fermented milk prepared in Example 11-1 reached adequate acidity. At the end of the fermentation, the number of lactic acid bacteria in the product was measured and the result is shown in Table 27 below. As a comparative example, coconut fermented milk was prepared in the same manner as in Example 10-1, except that the fermentation was performed using Y1 and Y2 strains (vegan-certified ‘starters for vegan fermented milk’), which are commercial strains available for fermented milk production, instead of the CAB701 strain.

TABLE 27

| | Lactobacillus count (1 × 10⁶ CFU/mL) | | |
|---|---|---|---|
| Acidity | CAB701 | Y1 | Y2 |
| 2.65 | 156 | 160 | 102 |
| 2.5 | 280 | 302 | 189 |

Looking at Table 27, it can be seen that the fermented milk with an acidity of 2.5 has about twice the number of lactobacilli as compared to the fermented milk with an acidity of 2.65. Furthermore, the fermented milk fermented with Y1 had the largest number of lactic acid bacteria, and the fermented milk fermented with the CAB701 strain of the present disclosure also had similar levels of lactic acid bacteria. On the other hand, the fermented milk fermented with Y2 satisfied the criteria for fermented milk but had a relatively low lactobacillus count.

### 11-3-2: Lactobacillus Count Over Time

Lactic acid bacteria starters (seed bacteria) used in the production of fermented milk are known to be the most influential factor on the quality of the product, including the product's physicochemical properties, lactic acid bacteria count, etc. Therefore, the change in the lactic acid bacteria count of the coconut fermented milk fermented using the CAB701 strain of the present disclosure and the commercial strains Y1 and Y2 were determined while storing at 4° C. The result is shown in Table 28 below.

TABLE 28

| | Lactobacillus count (1 × 10⁶ CFU/mL) | | |
|---|---|---|---|
| Time of analysis | CAB701 | Y1 | Y2 |
| Day 0 after preparation | 148 | 132 | 127 |
| Day 7 after preparation | 134 | 95 | 90 |
| (intermediate storage date) | | | |
| Day 14 after preparation | 92 | 61 | 41 |
| (expiration date) | | | |
| Day 16 after preparation | 76 | 26 | 18 |
| (day 2 after expiration) | | | |

Referring to Table 28, it can be seen that the coconut fermented milk fermented with the CAB701 strain of the present disclosure has a higher viable Lactobacillus cell count overall as compared to the coconut fermented milk fermented with the commercial strains (Y1 and Y2 strains), and maintains a high viable Lactobacillus cell count even after the expiration date. The fermented milks were found to meet the legal standard (1×10⁶ CFU/mL) of Lactobacillus cell count during the shelf life (14 days after preparation).

From the above results, it can be seen that the vegan fermented milk prepared with the CAB701 strain of the present disclosure has a significantly slower decrease in the viable Lactobacillus cell count during the storage period as compared to the vegan fermented milk prepared with the conventional vegan fermented milk strains, indicating that the CAB701 strain of the present disclosure is useful for the preparation of vegan fermented milk.

Although the present disclosure has been described with the specific exemplary embodiments mentioned above, various modifications or variations may be made without departing from the spirit and scope of the present disclosure. In addition, the modifications or variations are also included in the scope of the appended claims.

### [Accession Number]

Depository institution: Korean Culture Center of Microorganisms (KCCM)
Accession number: KCCM 13360P
Date of deposition: 20230616

We claim:

1.-16. (canceled)

17. A method of preventing, improving or treating obesity and/or a disease caused by a decrease in immune function by administering a composition comprising Lactococcus lactis subsp. lactis CAB701 strain deposited under accession number KCCM 13360P, a culture thereof, a supernatant of the culture, a concentrate of the culture, a lysate thereof, or a fermented product thereof as an active ingredient to a subject in need thereof.

18. The method of claim 17, wherein the stain comprises a 16S rRNA base sequence represented by SEQ ID NO 1.

19. The method of claim 18, wherein the stain has acid resistance to pH 2-3, bile resistance to 0.1-1% bile acid, pancreatic juice resistance to 0.1-1% pancreatic juice, and the ability of intestinal adherence.

20. The method of claim 17, wherein the composition has lipase inhibitory activity.

21. The method of claim 17, wherein the composition promotes the production of nitric oxide (NO), which is an immune marker.

22. The method of claim 17, wherein the composition increases the expression of the TNF-α, IL-6, IL-1β, COX-2 or iNOS gene, which are immune markers.

23. The method of claim 17, wherein the disease caused by a decrease in immune function is one or more selected from a group consisting of an infectious disease, an allergic disease, and chronic fatigue.

24. A method of preparing an anti-obesity or immune-enhancing vegan fermented milk, comprising:
(1) a step of inoculating a Lactococcus lactis subsp. lactis CAB701 strain deposited under accession number KCCM 13360P to a plant-based milk substitute at 0.2 to 3% (v/v); and
(2) a step of fermenting the plant-based milk substitute inoculated with the strain to pH of 3.5 to 4.5 at 35 to 39° C. for 12 to 24 hours.

25. The method of claim 24, wherein the plant-based milk substitute is a milk substitute prepared from one or more plant-based source selected from coconut, soybean, almond, oats and rice.

26. The method of claim 24, wherein the plant-based milk substitute has a solid content of 4 to 15 wt %.

27. The method of claim 24, wherein the vegan fermented milk has a number of live bacteria of 130×10⁶ CFU/mL or greater immediately after preparation and maintains a number of live bacteria of 70×10⁶ CFU/mL or greater up to 14 days after the preparation when stored at 1 to 8° C.


SEQUENCE LISTING (1 sequence)

SEQ ID NO: 1 - RNA, 231 nt, Lactococcus lactis
  source 1..231: mol_type = rRNA
tgcaagttgagcgctgaaggttggtacttgtaccgactggatgagcagcgaacgggtgagtaacgcgtggggaatctgcctttgagcgggggacaacatttggaaacgaatgctaataccgcataacaactttaaacacaagttttaagtttgaaagatgcaattgcatcactcaaagatgatcccgcgttgtattagctagttggtgaggtaaaggctcaccaaggcgat